SEQUENCE LISTING (19 sequences)

SEQ ID NO: 1 - DNA, 1019 bp, homo sapiens
agtaagcagccagccattccaagtggttgacatgactttgtttaactttatttgtatttctggctggtgtgtttacagccaataggtcaaactatcagtcagtgtagggccctgagaagtcgggtatttaagagcatctaataggcacagaattgtgctccatactgcttaaactgttccctaagtgtccaatttggagaaaacacccacacgcaggataaccggcgagtgacgcggagtggctgcgagtccaagttatcactaacggatggggagcttgggctgggcacagtccagcgtactgaacccttcccccaccgtttcacctgcatacagaggtgtgtactgtcaaaaagcagcgcctccaagtctcttctggcactgtctggacttggatccgaggcagacgaggaagctgagaaaaccctggcgttgaccccgtggacctgggcgccccgggaaggccagcgcttggtccaggcaggcggggcctgtgcggtgaccaccctggtcctgaaaagtcccagccccgagcgccctccctcctagacctggaggcctggaacagccaggtggacgtcggcccacctttcttttctttccttcccattttcctaccacctcccaccccactccgccttccgggcaaaggcagccagatccacccaggacacattctttgtccttatccctctgtgctcgtcccacagcaagccagtcgcggtccaaggctccagaggctgtgcaggaggccgagctgggtggcgatcagcggcgggtccctgtccaaaacccagcagagccgccagggacgccccagacacagaaggcggggcgcggggagggtggggagaccacagcagtgaggcgcgcgagccgggaagtgaacgaggactgactcctgtcgcttccgtagccgccacggacgccagagccgggaaccctgacggcacttactgctgacaaacaacctgctccgtggagcgcctgaaacccaatctttgggtgagtcgcgcgac

SEQ ID NO: 2 - DNA, 173 bp, homo sapiens
agtaagcagccagccattccaagtggttgacatgactttgtttaactttatttgtatttctggctggtgtgtttacagccaataggtcaaactatcagtcagtgtagggccctgagaagtcgggtatttaagagcatctaataggcacagaattgtgctccatactgcttaaa

SEQ ID NO: 3 - DNA, 3850 bp, homo sapiens
cggcacgagcggcacgagagaagagactccaatcgacaagaagctggaaaagaatgatgttgtccttaaacaacctacagaatatcatctataacccggtaatcccgtttgttggcaccattcctgatcagctggatcctggaactttgattgtgatacgtgggcatgttcctagtgacgcagacagattccaggtggatctgcagaatggcagcagcgtgaaacctcgagccgatgtggcctttcatttcaatcctcgtttcaaaagggccggctgcattgtttgcaatactttgataaatgaaaaatggggacgggaagagatcacctatgacacgcctttcaaaagagaaaagtcttttgagatcgtgattatggtgctgaaggacaaattccaggtggctgtaaatggaaaacatactctgctctatggccacaggatcggcccagagaaaatagacactctgggcatttatggcaaagtgaatattcactcaattggttttagcttcagctcggacttacaaagtacccaagcatctagtctggaactgacagagatagttagagaaaatgttccaaagtctggcacgccccagcttagcctgccattcgctgcaaggttgaacacccccatgggccctggacgaactgtcgtcgttcaaggagaagtgaatgcaaatgccaaaagctttaatgttgacctactagcaggaaaatcaaaggatattgctctacacttgaacccacgcctgaatattaaagcatttgtaagaaattcttttcttcaggagtcctggggagaagaagagagaaatattacctctttcccatttagtcctgggatgtactttgagatgataatttattgtgatgttagagaattcaaggttgcagtaaatggcgtacacagcctggagtacaaacacagatttaaagagctcagcagtattgacacgctggaaattaatggagacatccacttactggaagtaaggagctggtagcctacctacacagctgctacaaaaaccaaaatacagaatggcttctgtgatactggccttgctgaaacgcatctcactggtcattctattgtttatattgttaaaatgagcttgtgcaccattaggtcctgctgggtgttctcagtccttgccatgacgtatggtggtgtctagcactgaatggggaaactgggggcagcaacacttatagccagttaaagccactctgccctctctcctactttggctgactcttcaagaatgccattcaacaagtatttatggagtacctactataatacagtagctaacatgtattgagcacagattttttttggtaaatctgtgaggagctaggatatatacttggtgaaacaaaccagtatgttccctgttctcttgagcttcgactcttctgtgcgctactgctgcgcactgctttttctacaggcattacatcaactcctaaggggtcctctgggattagttatgcagatattaaatcacccgaagacactaacttacagaagacacaactccttccccagtgatcactgtcataaccagtgctctgccgtatcccatcactgaggactgatgttgactgacatcattttctttatcgtaataaacatgtggctctattagctgcaagctttaccaagtaattggcatgacatctgagcacagaaattaagccaaaaaaccaaagcaaaacaaatacatggtgctgaaattaacttgatgccaagcccaaggcagctgatttctgtgtatttgaacttacccgaaatcagagtctacacagacgcctacagaagtttcaggaagagccaagatgcattcaatttgtaagatatttatggccaacaaagtaaggtcaggattagacttcaggcattcataaggcaggcactatcagaaagtgtacgccaactaagggacccacaaagcaggcagaggtaatgcagaaatctgttttgttcccatgaaatcaccaatcaaggcctccgttcttctaaagattagtccatcatcattagcaactgagatcaaagcactcttccactttacgtgattaaaatcaaacctgtatcagcaagttaaatggttccatttctgtgatttttctattatttgaggggagttggcagaagttccatgtatatgggatctttacaggtcagatcttgttacaggaaatttcaaaggtttgggagtggggagggaaaaaagctcagtcagtgaggatcattccacattagactggggcagaactctgccaggatttaggaatattttcagaacagattttagatattatttctatccatatattgaaaaggaataccattgtcaatcttatttttttaaaagtactcagtgtagaaatcgctagcccttaattcttttccagcttttcatattaatgtatgcagagtctcaccaagctcaaagacactggttgggggtggagggtgccacagggaaagctgtagaaggcaagaagactcgagaatcccccagagttatctttctccataaagaccatcagagtgcttaactgagctgttggagactgtgaggcatttaggaaaaaaatagcccactcacatcattccttgtaagtcttaagttcattttcattttacgtggaggaaaaaaatttaaaaagctattagtatttattaatgaattttactgagacatttcttagaaatatgcacttctatactagcaagctctgtctctaaaatgcaagttggccttttgcttgccacatttctgcattaaacttctatattagcttcaaaggcttttaatctcaatgcgaacattctacgggatgttcttagatgcctttaaaaagggggcaagatctaattttatttgaaccctcactttccaactttcaccatgacccagtactagagattagggcacttcaaagcattgaaaaaaatctactgatacttactttcttagacaagtagttcttagttaaccaccaatggaactgggttcattctgaatcctggaggagcttcctcgtgccacccagtgtttctgggccctctgtgtgagcagccaggtgtgagctgttttagaagcagcgtgttgccttcatctctcccgtttcccaaaagaacaaaggataaaggtgacagtcacactcctgggttaaaaaaagcattccagaaccacttctctttatgggcacaacaacaaagaagctaagttcgcctacccaaatgaaagtaggctttacagtcaagtacttctgttgattgctaaataacttcattttcttgaaatagagcaactttgagtgaaatctgcaacatggataccatgtatgtaagatactgctgtacagaagagttaaggcttacagtgcaaatgaggcgtcagctttgggtgctaaaattaacaagtctaatattattaccatcaatcaggaagagataataaatgtttaaacaaacacagcagtctgtataaaaatacgtgtatatttactctttctgtgcacgctctatagcataggcaggagaggcttatgtggcagcacaagccaggtggggattttgtaaagaagtgataaaacatttgtaagtaatccaagtaggagatattaaggcaccaaaagtaacatggcacccaacacccaaaaataaaaatatgaaatatgagtgtgaactctgagtagagtatgaaacaccacagaaagtcttagaaatagctctggagtggctctcccaggacagtttccagttggctgaatagtcttttggcactgatgttctacttcttcacattcatctaaaaaaaaaaaaaaaaaa

SEQ ID NO: 4 - DNA, 126 bp, homo sapiens
cctagtaatagaggaggagacatttctaaaatcgcacccagaactgtctacaccaagagcaaagattcgactgtcaatcacactttgacttgcaccaaaataccacctatgaactatgtgtcaaag

SEQ ID NO: 5 - DNA, 132 bp, homo sapiens
cagactgtctctcccctcctgggatttacagggtcatggctctgaaacattctgtagtgttctttggacacgagttttccctggagatcgctttctgcaggcctattggtcctgactgtggcttcttttcag

SEQ ID NO: 6 - protein, 317 aa, homo sapiens
MMLSLNNLQNIIYNPVIPFVGTIPDQLDPGTLIVIRGHVPSDADRFQVDLQNGSSVKPRADVAFHFNPRFKRAGCIVCNTLINEKWGREEITYDTPFKREKSFEIVIMVLKDKFQVAVNGKHTLLYGHRIGPEKIDTLGIYGKVNIHSIGFSFSSDLQSTQASSLELTEIVRENVPKSGTPQLSLPFAARLNTPMGPGRTVVVQGEVNANAKSFNVDLLAGKSKDIALHLNPRLNIKAFVRNSFLQESWGEEERNITSFPFSPGMYFEMIIYCDVREFKVAVNGVHSLEYKHRFKELSSIDTLEINGDIHLLEVRSW

SEQ ID NO: 7 - protein, 131 aa, homo sapiens
FAARLNTPMGPGRTVVVQGEVNANAKSFNVDLLAGKSKDIALHLNPRLNIKAFVRNSFLQESWGEEERNITSFPFSPGMYFEMIIYCDVREFKVAVNGVHSLEYKHRFKELSSIDTLEINGDIHLLEVRSW

SEQ ID NO: 8 - protein, 183 aa, homo sapiens
MMLSLNNLQNIIYNPVIPFVGTIPDQLDPGTLIVIRGHVPSDADRFQVDLQNGSSVKPRADVAFHFNPRFKRAGCIVCNTLINEKWGREEITYDTPFKREKSFEIVIMVLKDKFQVAVNGKHTLLYGHRIGPEKIDTLGIYGKVNIHSIGFSFSSDLQSTQASSLELTEIVRENVPKSGTPQL

SEQ ID NO: 9 - protein, 15 aa, homo sapiens
MLSLNNLQNIIYNPV

SEQ ID NO: 10 - protein, 29 aa, homo sapiens
IPFVGTIPDQLDPGTLIVIRGHVPSDADR

SEQ ID NO: 11 - protein, 70 aa, homo sapiens
FQVDLQNGSSVKPRADVAFHFNPRFKRAGCIVCNTLINEKWGREEITYDTPFKREKSFEIVIMVLKDKFQ

SEQ ID NO: 12 - protein, 40 aa, homo sapiens
VAVNGKHTLLYGHRIGPEKIDTLGIYGKVNIHSIGFSFSS

SEQ ID NO: 13 - protein, 19 aa, homo sapiens
DLQSTQASSLELTEIVREN

SEQ ID NO: 14 - protein, 9 aa, homo sapiens
VPKSGTPQL

SEQ ID NO: 15 - protein, 30 aa, homo sapiens
SLPFAARLNTPMGPGRTVVVQGEVNANAKS

SEQ ID NO: 16 - protein, 55 aa, homo sapiens
FNVDLLAGKSKDIALHLNPRLNIKAFVRNSFLQESWGEEERNITSFPFSPGMYFE

SEQ ID NO: 17 - protein, 49 aa, homo sapiens [sequence omitted]

SEQ ID NO: 18 - DNA, 549 bp, homo sapiens
atgatgttgtccttaaacaacctacagaatatcatctataacccggtaatcccgtttgttggcaccattcctgatcagctggatcctggaactttgattgtgatacgtgggcatgttcctagtgacgcagacagattccaggtggatctgcagaatggcagcagcgtgaaacctcgagccgatgtggcctttcatttcaatcctcgtttcaaaagggccggctgcattgtttgcaatactttgataaatgaaaaatggggacgggaagagatcacctatgacacgcctttcaaaagagaaaagtcttttgagatcgtgattatggtgctgaaggacaaattccaggtggctgtaaatggaaaacatactctgctctatggccacaggatcggcccagagaaaatagacactctgggcatttatggcaaagtgaatattcactcaattggttttagcttcagctcggacttacaaagtacccaagcatctagtctggaactgacagagatagttagagaaaatgttccaaagtctggcacgccccagctt

SEQ ID NO: 19 - DNA, 832 bp, homo sapiens
ctgttccctaagtgtccaatttggagaaaacacccacacgcaggataaccggcgagtgacgcggagtggctgcgagtccaagttatcactaacggatggggagcttgggctgggcacagtccagcgtactgaacccttcccccaccgtttcacctgcatacagaggtgtgtactgtcaaaaagcagcgcctccaagtctcttctggcactgtctggacttggatccgaggcagacgaggaagctgagaaaaccctggcgttgaccccgtggacctgggcgccccgggaaggccagcgcttggtccaggcaggcggggcctgtgcggtgaccaccctggtcctgaaaagtcccagccccgagcgccctccctcctagacctggaggcctggaacagccaggtggacgtcggcccacctttcttttctttccttcccattttcctaccacctcccaccccactccgccttccgggcaaaggcagccagatccacccaggacacattctttgtccttatccctctgtgctcgtcccacagcaagccagtcgcggtccaaggctccagaggctgtgcaggaggccgagctgggtggcgatcagcggcgggtccctgtccaaaacccagcagagccgccagggacgccccagacacagaaggcggggcgcggggagggtggggagaccacagcagtgaggcgcgcgagccgggaagtgaacgaggactgactcctgtcgcttccgtagccgccacggacgccagagccgggaaccctgacggcacttactgctgacaaacaacctgctccgtggagcgcctgaaacccaatctttgg

(19) United States
(12) Patent Application Publication
(10) Pub. No.: US 2004/0235769 A1
(43) Pub. Date: Nov. 25, 2004

(54) Anti-tumor effects of prostage Carcinoma Tumor Antigen-1

(76) Inventors: Paul B. Fisher, Scarsdale, NY (US); Rahul V. Gopalkrishnan, New York, NY (US)

Correspondence Address: BAKER & BOTTS, 30 ROCKEFELLER PLAZA, NEW YORK, NY, 10112

(21) Appl. No.: 10/795,927

(22) Filed: Mar. 8, 2004

Related U.S. Application Data
(63) Continuation of application No. PCT/US02/28587, filed on Sep. 6, 2002.
Application No. PCT/US02/28587 is a continuation-in-part of application No. 09/948,227, filed on Sep. 7, 2001.

(51) Int. Cl. A61K 48/00; A61K 38/19

(52) U.S. Cl. 514/44; 424/85.1

(57) ABSTRACT

The present invention relates to methods of inhibiting the proliferation and/or metastasis of a cancer cell by administering, to the cancer cell, a molecule which increases, in the cell or at the cell surface, the amount of a Bivalent Prostate Carcinoma Tumor Antigen-1 (“B-PCTA-1”) protein (referred to as “bivalent” because it comprises both carbohydrate recognition domains (“CRDs”)). It is based, at least in part, on the discovery that increased expression of the full-length open reading frame of the PCTA-1 gene suppressed proliferation of tumor cells in soft agar (a characteristic associated with malignancy and tumor metastasis), whereas increased expression of a PCTA-1 gene lacking the second CRD-encoding region had the opposite effect, increasing the anchorage-independent proliferation of the tumor cells.

The invention described herein was supported in part by National Institutes of Health Grant CA74468, so that the United States government has certain rights herein.

### 1. INTRODUCTION

The present invention relates to methods of inhibiting the proliferation and/or metastasis of a tumor cell by administering, to the tumor cell, a molecule which increases the amount of Prostate Carcinoma Tumor Antigen-1 (“PCTA-1”) protein in the cell or at the cell surface. It is based, at least in part, on the discovery that PCTA-1, previously identified as a tumor associated antigen, has tumor suppressive properties.

### 2. BACKGROUND OF THE INVENTION

In vertebrates, galectins are a family of proteins presently known to include 12 polypeptides (Colnot et al., 1996, Biochem. Soc. Trans. 24:141-146; Cooper and Barondes, 1999, Glycobiology 9: 979-984; Hughes, 1997, Biochem. Soc. Trans. 25: 1194-1198; Kasai and Hirabayashi, 1996, J. Biochem. (Tokyo) 119: 1-8; Hotta et al., 2001, J. Biol. Chem. 276:34089-34097) encoded by distinct genes. These proteins are structurally related, containing conserved domains that give them the ability to recognize and bind glycoproteins having β-galactoside side-chain residues (Rini, 1995, Curr. Opin. Struct. Biol. 5: 617-621).

Within the galectin gene family, further sub-classification can be made by distinguishing between “prototype” galectins, which contain a single carbohydrate recognition domain (“CRD”), “tandem repeat” galectins, which contain two CRD domains separated by a linker sequence, and chimeric-type proteins wherein an unrelated amino-terminal domain is linked to a CRD (Cooper and Barondes, 1999, Glycobiology 9: 979-984; Hughes, 1997, Biochem. Soc. Trans. 25: 1194-1198).

Galectins appear to have originated at a fairly early time during evolution since they occur in marine sponges and fungi (Arata et al., 1997, J. Biochem. (Tokyo) 121: 1002-1009; Cooper et al., 1997, J. Biol. Chem. 272: 1514-1521; Greenhalgh et al., 1999, Mol. Biochem. Parasitol. 98: 285-289; Wagner-Hulsmann et al., 1996, Glycobiology 6: 785-793). Functionally, the galectins as a family have been associated with diverse phenomena in every cellular compartment, including the cell surface and extracellular roles in adhesion (Inohara and Raz, 1995, Cancer Res. 55: 3267-3271; Kaltner and Stierstorfer, 1998, Acta Anat. 161: 162-179; Lotan et al., 1994, Glycoconj. J. 11: 462-468), cell to cell recognition and signaling (Inohara and Raz, 1995, Cancer Res. 55: 3267-3271), intracellular association with specific organelles and within the nucleus in association with components of the splicing machinery and with mRNA splicing (Dagher et al., 1995, Proc. Natl. Acad. Sci. U.S.A. 92: 1213-1217; Vyakarnam et al., 1997, Mol. Cell. Biol. 17: 4730-4737; Vyakarnam et al., 1998, Exp. Cell. Res. 242: 419-428). Incompletely defined functional associations of several galectins have also been reported in embryonic development, signal transduction, differentiation (Lu et al., 1998, Biol. Chem. 379: 1323-1331; Lu and Lotan, 1999, Biochim. Biophys. Acta 1444: 85-91), transformation (Bresalier et al., 1997, Cancer 80: 776-787; Ellerhorst et al., 1999, Int. J. Oncol. 14: 217-224; Gillenwater et al., 1996, Head Neck 18: 422-432), tumor suppression, metastasis and the immune response (Akahani et al., 1997, Cancer Res. 57: 5272-5276; Barondes et al., 1994, J. Biol. Chem. 269: 20807-20810; Bresalier et al., 1996, Cancer Res. 56: 4354-4357; Bresalier et al., 1997, Cancer 80: 776-787; Chammas et al., 1996, Braz. J. Med. Biol. Res. 29: 1141-1149; Colnot et al., 1996, Biochem. Soc. Trans. 24: 141-146; Cortegano et al., 1998, J. Immunol. 161: 385-389; Hebert et al., 1996, CR Acad. Sci. III 319: 871-877; Hsu et al., 1999, Int. J. Cancer 81: 519-526; Remmelink et al., 1999, J. Cancer Res. Clin. Oncol. 125: 275-285).

At the present time, the normal physiological roles of galectins remain substantially undefined and considerable effort is being devoted towards understanding their biological relevance in specific cellular contexts. Mammalian galectin nomenclature broadly reflects the temporal order of discovery; galectin-1 and -3, therefore, find the highest degree of representation in the literature. Galectins containing tandemly repeated CRD domains, including galectins-4, -6, -8 and -9, remain relatively uncharacterized. It is believed that this type of galectin arose by duplication and subsequent divergence of relevant exons from single CRD-containing genes during evolution. An average of 30% amino acid identity and conserved structural homology between the two CRDs substantiate this notion (Hadari et al., 1995, J. Biol. Chem. 270: 3447-3453; Wada and Kanwar, 1997, J. Biol. Chem. 272: 6078-6086).

Galectins, in general, have the propensity to form higher order multimers, usually as a result of binding to glycoconjugate receptors. In the native unbound state, single CRD galectins (galectins-1 and -2) exist as dimers while galectins-3, -7 and -10 form higher order aggregates only in the presence of receptors with multiple binding sites (Arata et al., 1997, J. Biochem. (Tokyo) 121: 1002-1009; Chammas et al., 1996, Braz. J. Med. Biol. Res. 29: 1141-1149; Cho and Cummings, 1995, J. Biol. Chem. 270: 5198-5206; Cho and Cummings, 1995, J. Biol. Chem. 270: 5207-5212; Kaltner and Stierstorfer, 1998, Acta Anat. 161: 162-179).

Double CRD-containing molecules, through the ability of each CRD to recognize distinct glycoconjugates, are thought to have the additional capability of acting as heterobifunctional crosslinking agents and therefore have a broadened range of interactive capacity (Arata et al., 1997, J. Biochem. (Tokyo) 121: 1002-1009; Gitt et al., 1998, J. Biol. Chem. 273: 2954-2960; Kaltner and Stierstorfer, 1998, Acta Anat. 161: 162-179). This implies that a further level of complexity could exist in the range of interactions in which tandem repeat type galectins can participate.

PCTA-1 was isolated in a cloning project to identify molecules specifically expressed on the surface of prostate cancer cells. This approach involved an immunological subtraction scheme, surface epitope-masking (SEM), in which polyclonal antibodies produced against unmodified cloned rat embryo fibroblast cells were used to coat the surface of genetically modified cells transformed using high molecular weight DNA from a human prostate cancer cell line, LNCAP. The coated cells were used to raise monoclonal antibodies which recognize prostate cancer specific surface epitopes (Shen et al., 1994, J. Natl. Cancer Inst. 86: 91-98, U.S. Pat. No. 5,851,764 by Fisher et al., issued Dec. 22, 1998). Expression screening of a cDNA library to identify clones encoding the antigen recognized by the respective antibody was performed to determine the identity of the reactive antigen. One of the several monoclonal antibodies isolated in the course of this screen, designated as Pro 1.5, specifically recognized a cDNA clone expressing a protein having 81% amino acid sequence homology to a sequence present in the databases at that time, namely rat galectin-8 (Su et al., 1996, Proc. Natl. Acad. Sci. U.S.A. 93: 7252-7257). Based on this homology, it appeared that the isolated sequence was a human tumor homolog of the rat sequence. Antibody studies and RT-PCR based expression analysis in prostate cancer cell lines and tissue samples indicated an association of PCTA-1 expression with prostate carcinoma (Su et al., 1996, Proc. Natl. Acad. Sci. U.S.A. 93: 7252-7257). Similarly, a correlation between presence of PCTA-1 RNA and metastatic cancer has been drawn (U.S. Pat. No. 6,255,049 by Fisher, issued Jul. 3, 2001).

The present invention relates to the surprising discovery that increased expression of PCTA-1, hitherto believed to be a promoter of the oncogenic process, can suppress certain malignant characteristics in cancer cells.

### 3. SUMMARY OF THE INVENTION

The present invention relates to methods of inhibiting the proliferation and/or metastasis of a cancer cell by administering, to the cancer cell, a molecule which increases, in the cell or at the cell surface, the amount of a Bivalent Prostate Carcinoma Tumor Antigen-1 (“B-PCTA-1”) protein (referred to as “bivalent” because it comprises both carbohydrate recognition domains (“CRDs”)). It is based, at least in part, on the discovery that increased expression of the full-length open reading frame of the PCTA-1 gene suppressed proliferation of tumor cells in soft agar (a characteristic associated with malignancy and tumor metastasis), whereas increased expression of a PCTA-1 gene lacking the second CRD-encoding region had the opposite effect, increasing the anchorage-independent proliferation of the tumor cells.

Accordingly, in various embodiments, the present invention provides for inhibiting the transformed phenotype of cancer cells, inhibiting cancer cell proliferation, inhibiting cancer cell metastasis, and treating cancer in human and non-human subjects utilizing methods and compositions which promote increased intracellular and/or extracellular levels of B-PCTA-1 protein. In particular embodiments, the methods further comprise administering, to the cancer cell and/or subject, a differentiation promoting agent.

In alternative embodiments, the present invention relates to the oncogenic properties of truncated PCTA-1 (“T-PCTA-1”) protein. In this regard, the invention provides for diagnostic methods, wherein the presence of T-PCTA-1 is a marker for malignancy, and for methods of producing model tumor cell systems.

### 4. BRIEF DESCRIPTION OF THE FIGURES

FIG. 1A-C. Genomic structure of PCTA-1. (A) Exons encoding the PCTA-1 mRNA are represented approximately to scale by open rectangles. Designation is indicated within or adjoining to and size in bp below the respective exon. Translation start and stop codon positions, the 5' and 3' UTRs and regions of the gene encoding conserved structural or functional elements present in the protein shown are above the relevant exons. CRD1 is referred to herein as the “first” CRD, and CRD2 is referred to herein as the “second” CRD. The non-coding or coding portion of an exon containing mixed UTR and coding sequence in the same exon and two known alternative spliced exons are highlighted by curly parentheses. (B) The 5′ and 3′ exonic sequences at splice junction boundaries has been tabulated with the corresponding exon number and coordinates of the cDNA sequence. (C) Alternate exons 1 (7′) and 2 (7″). (D) Amino acid sequence of PCTA-1 (SEQ ID NO:6) showing exon boundaries and subpeptides SEQ ID NOS: 9-17. (E). Amino acid sequence in the approximate region encoding the second CRD (SEQ ID NO:7).

FIG. 2A-D. (A) Construct for producing truncated PCTA-1 (“T-PCTA-1”). The end of the CRD-1 deletion is at amino acid 186; a UAG stop codon was introduced by PCR primer. (13) Amino acid sequence (SEQ ID NO:8) of the T-PCTA-1 encoded by the construct in (A); (C) nucleic acid (SEQ ID NO:18) and amino acid (SEQ ID NO:8) sequence of the T-PCTA-1 shown in (A); and (D) construct encoding B-PCTA-1.

FIG. 3A-B. Transcription start site and primer extension analysis of the first exon, as contained in the sequence shown in (A) (SEQ ID NO:1), which further indicates the antisense extension primer (in light gray typeset), the +1 transcription initiation site and the putative “TATA-box” promoter element based on the position of extension primer size and size of the extension products. Also shown are the splice donor and acceptor sites (in bold) of an alternatively spliced form of the 5′ UTR. (B) Primer extension analysis using the antisense extension primer shown in (A) generated two extension products (lane 1). An alternatively processed form of the 5′ UTR, reported under GenBank Accession numbers AF074000, AF074001 and AF074002 (smaller product in lane 1) is generated by splicing the intervening sequences between positions 572-914. As discussed in the text, using RT-PCR analyses with prostate cancer cell RNA (PC-3 total RNA, lane 1) we primarily detected the unprocessed longer form of the UTR. Specificity of the extension product was determined by using yeast total RNA as negative control (lane 2). Size and sequence of the transcription start site was determined by running sequencing reactions with the primer extension primer (not shown) and end-labeled commercial size standard (lane M).

FIG. 4A-G. Human tissue mRNA expression pattern and variant forms of PCTA-1 mRNA compared to human galectin-3 expression. Commercial multiple tissue Northern blots (Clontech) were probed sequentially with radiolabeled PCTA-1 ORF probe (A-C, lanes 1-23), 3′ UTR probe (D, lanes 24-31) and human galectin-3 ORF probe (E-G, lanes 1±23). Lanes 1-8 in A and E contain mRNA from: lane 1=heart; lane 2=whole brain; lane 3=placenta; lane 4=lung; lane 5=liver; lane 6=skeletal muscle; lane 7=kidney; lane 8=pancreas. Lanes 9-16 in B and F and lanes 24-31 in D contain mRNA from: lanes 9 and 24=spleen; lanes 10 and 25=thymus; lanes 11 and 26=prostate; lanes 12 and 27=testis; lanes 13 and 28=ovary; lanes 14 and 29=small intestine; lanes 15 and 30=colon; lanes 16 and 31=peripheral blood lymphocytes. Lanes 17-23 (C and G) contain mRNA from: lane 17=stomach; lane 18=thyroid; lane 19=spinal cord; lane 20=lymph node; lane 21=trachea, lane 22=adrenal gland; and lane 23=bone marrow, respectively.

FIG. 5A-H. Multiple forms of PCTA-1 message in prostate and melanoma. (A-D) show Northern blots using prostate-derived RNA. Total RNA from human prostate cancer cell lines DU-145 (lanes marked “D”), LNCaP (lanes marked “L”), PC-3 (lanes marked “P”) and commercial (Clontech) normal prostate (lanes marked “N”) was analyzed by Northern blot using region specific cDNA probes containing: (A) the ORF, (1) alternate exon 1 (ALTA), (C) alternate exon 2 (ALTA) and (D) 3′ UTR. Four identical sets of RNA were run in parallel on the same gel, transferred and strips cut and individually probed for each set. A commercial RNA standard (Life Technologies, (E) A representative ethidium bromide (EtBr) stained RNA gel before transfer is shown to indicate approximately equivalent RNA was analyzed from each source; lane marked “M”) was used to determine size of signal obtained after autoradiographic exposure. (F-G) show Northern blots using melanoma-derived RNA. Total RNA from human melanoma cell lines C8161 (C) and HO-1 (H) with normal prostate RNA (N) as control was analyzed by Northern blot using region specific cDNA probes containing (F) the ORF or (G) the 3′ UTR. (H) The ethidium bromide (EtBr) stained RNA gel before transfer is shown to indicate approximately equivalent RNA was analyzed from each of these sources. A commercial RNA standard (Life Technologies, Lane M) was used to determine size of signal obtained after autoradiographic exposure.

FIG. 6. Schematic representation of various RNA isoforms of PCTA-1. Discrete blocks of sequence elements that contribute to observed and predicted mRNA isoforms of PCTA-1 are shown for ease of representation and correlation with the known exon-intron structure and poly adenylation pattern. The shorter processed form of 5′ UTR (FIG. 1) is represented by two lightly filled-in adjoining rectangles (internally spliced short 5′ UTR) as opposed to the full non-spliced 5′ UTR (long form of 5′ UTR). Since the ATG containing exon is also comprised of a small and invariant portion of 5′ UTR sequence (FIG. 1), these have been shown together as a single unit, the bulk of which comprises the ORF. The variable forms of this unit contains either of three possibilities, inclusion of two additional exons which increases the length of the ORF and a third probably predominant form, lacking either alternate exon formed by direct splicing of exons 7-8 (FIG. 1). The three differentially processed 3′ UTRs (dark shaded rectangles) make up the mature transcripts. The final sizes of each possible transcript are indicated in the right hand column depending on which specific combination of elements (middle columns) are contained within them.

FIG. 7A-B. Intracellular localization of PCTA-1. (A) Western blot with anti-GFP monoclonal antibody of total cell extracts, from HeLa cells transiently transfected with GFP or GFP-PCTA-1 fusion expressing plasmid constructs. The 35 kDa GFP band is shifted up by an additional 36 kDa encoded by the PCTA-1 ORF in the fusion protein as determined by protein molecular weight standards (not shown). (B) Fluorescence microscopy of HeLa cells expressing the PCTA-1 GFP fusion protein seen in (A). Cells transfected in parallel with non-fusion GFP showed a uniform distribution in nucleus and cytoplasm (not shown).

FIG. 8A-B. Phenotypic effect of PCTA-1 expression in human cell lines. (A) Colony formation assay of clonally isolated HeLa cells stably expressing full-length (PCTA-1 FL), deleted ORF containing only the first CRD (PCTA-1 CR1) or vector control (VECTOR). The number of colonies formed by vector was taken as 100% to determine extent of relative colony forming ability of cells expressing full-length or truncated PCTA-1. The assay was performed twice in quadruplicate for each point. (B) Colony formation assay of DU-145 cells infected with non-replicating Adenovirus vector control (VECTOR), Adenovirus expressing full-length PCTA-1 (PCTA-1 FL) and uninfected cells (DU-145 WT). The number of colonies formed at the end of 3 weeks was counted for each set performed in quadruplicate. (Gopalkrishnan et al., 2000, Oncogene 19:4405-4416).

FIG. 9. Nucleic acid sequence of PCTA-1 encoding cDNA, as contained in GenBank Accession No. L78132 and SEQ ID NO: 3. The coding region extends between nucleotides 54 and 1004.

FIG. 10. Amino acid sequence of PCTA-1 (SEQ ID NO:6).

FIG. 11. PCR-based genotyping of transgenic mice derived from crosses of singly transgenic TRAMP and B-PCTA-1 mice. T indicates PCR reactions with primers specific for the TRAMP transgene. P indicates PCR reactions with primers specific for the B-PCTA-1 transgene. In this screening, the animal from which the DNA used as an amplification template for the PCR reactions shown in lane 1 was doubly transgenic, while all other animals tested were singly transgenic.

FIG. 12. Frequency of singly-and doubly-transgenic male mice generated through crosses of singly transgenic TRAMP and B-PCTA-1 mice.

FIG. 13. Infiltration of abdominal cavity of TRAMP transgenic mouse by prostate-derived adenocarcinoma.

### 5. DETAILED DESCRIPTION OF THE INVENTION

For clarity of presentation, and not by way of limitation, the detailed description of the invention is divided into the following subsections:

(a) PCTA-1 nucleic acid molecules;

(b) PCTA-1 proteins and antibodies;

(c) methods of inhibiting cancer cell proliferation and/or metastasis;

(d) methods of inhibiting the expression of an oncogenic PCTA-1 protein;

(e) methods of diagnosing malignancy, and

(f) preparation of model systems.

### 5.1 PCTA-1 Nucleic Acid Molecules

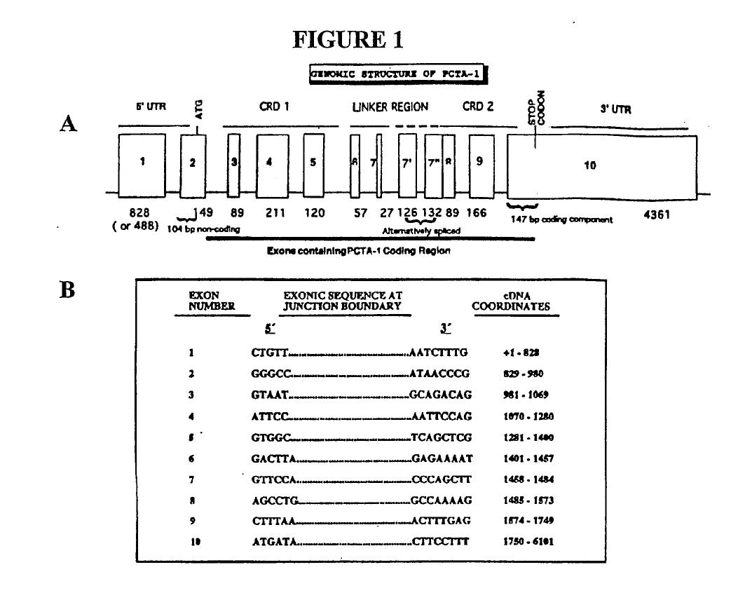

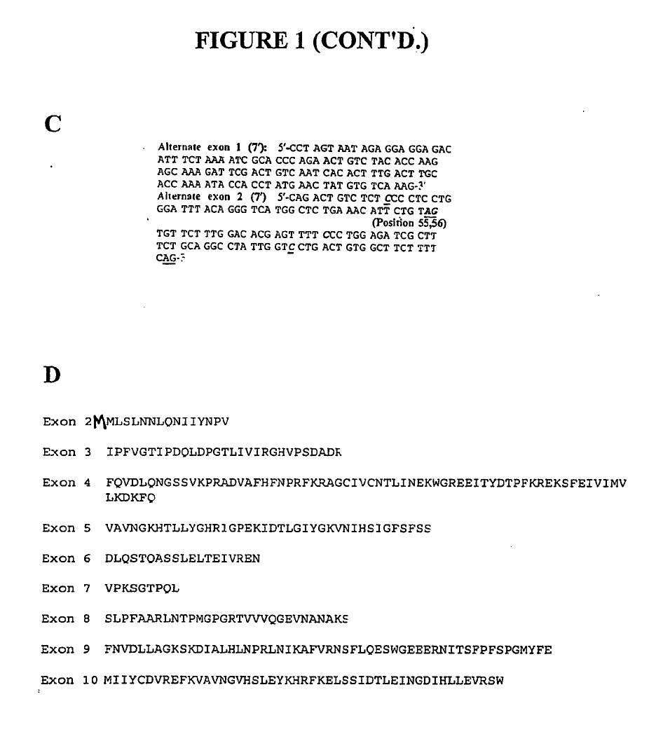

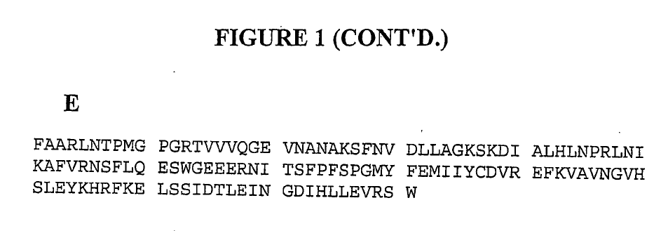

The present invention relates to compositions and/or methods which contain and/or utilize PCTA-1 nucleic acid molecules as comprised in the PCTA-1 gene, as schematically depicted in FIG. 1 and located at human chromosome region 1q42-43. The nucleic acid molecules of the invention may or may not comprise protein-encoding sequence. Nucleic acids may be DNA or RNA, and may comprise modified bases.

Thus, the invention provides for nucleic acid molecules including the following, taken singly or in combination (all of which are referred to herein as “PCTA-1 nucleic acid molecules”):

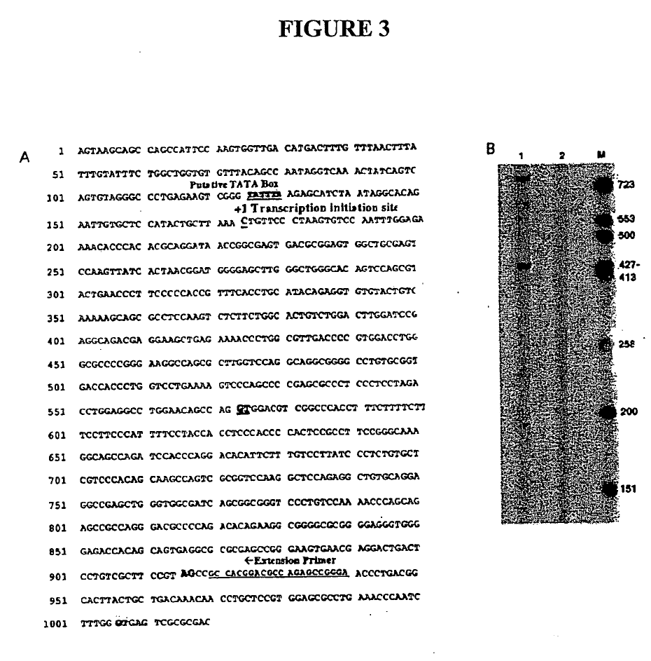

(i) sequence upstream of exon 1, comprising a sequence as set forth in FIG. 3A from residue 1 through residue 173 (SEQ ID NO:2);

(ii) exon 1, having cDNA coordinates from 1-828, where the cDNA is depicted as open boxes in FIG. 1, comprising a sequence as set forth in FIG. 3A from residue 174 through residue 1004 (SEQ ID) NO:19);

(iii) intron 1, as comprised in the genomic sequence between the 3′ border of exon 1 (5′-AATCTTTG-3′) and the 5′ border of exon 2 (5′-GGGCC-3′);

(iv) exon 2, having cDNA coordinates from 829-980, comprising residues 19-98 of the nucleic acid sequence set forth in GenBank Accession No. L78132 and SEQ ID NO:3, including the initiation codon at residue 54;

(v) intron 2, as comprised in the genomic sequence between the 3′ border of exon 2 (5′-ATAACCCG-3′) and the 5′ border of exon 3 (5′-GTAAT-3′);

(vi) exon 3, having cDNA coordinates from 981-1069, comprising residues 99-187 of the nucleic acid sequence set forth in GenBank Accession No. L78132 and SEQ ID NO:3;

(vii) intron 3, as comprised in the genomic sequence between the 3′ border of exon 3 (5′-GCAGACAG-3) and the 5′ border of exon 4 (5′-ATTCC-3′);

(viii) exon 4, having cDNA coordinates from 1070-1280, comprising residues 188-398 of the nucleic acid sequence set forth in GenBank Accession No. L78132 and SEQ ID NO:3;

(ix) intron 4, as comprised in the genomic sequence between the 3′ border of exon 4 (5′-AATTCCAG-3′) and the 5′ border of exon 5 (5′-GTGGC-3′);

(x) exon 5, having cDNA coordinates from 1281-1400, comprising residues 399-518 of the nucleic acid sequence set forth in GenBank Accession No. L78132 and SEQ ID NO:3;

(xi) intron 5, as comprised in the genomic sequence between the 3′ border of exon 5 (5′-TCAGCTCG-3′) and the 5′ border of exon 6 (5′-GACTTA-3);

(xii) exon 6, having cDNA coordinates from 1401-1457, comprising residues 519-575 of the nucleic acid sequence set forth in GenBank Accession No. L78132 and SEQ ID NO:3;

(xiii) intron 6, as comprised in the genomic sequence between the 3′ border of exon 6 (5′-GAGAAAAT-3′) and the 5′ border of exon 7 (5′-GTTCCA-3′);

(xiv) exon 7, having cDNA coordinates from 1458-1484, comprising residues 576-602 of the nucleic acid sequence set forth in GenBank Accession No. L78132 and SEQ ID NO:3;

(xv) intron 7, as comprised in the genomic sequence between the 3′ border of exon 7 (5′-CCCAGCTT-3′) and the 5′ border of exon 8 (5′-AGCCTG-3′), where intron 7 comprises alternate exons 1 (“7′”) and 2 (“7″”), having sequences 5′-CCT AGT AAT AGA GGA GGA GAC ATT TCT AAA ATC GCA CCC AGA ACT GTC TAC ACC AAG AGC AAA GAT TCG ACT GTC AAT CAC ACT TTG ACT TGC ACC AAA ATA CCA CCT ATG AAC TAT GTG TCA AAG-3′ (SEQ ID NO: 4) and 5′-CAG ACT GTC TCT CCC CTC CTG GGA TTT ACA GGG TCA TGG CTC TGA AAC ATT CTG TAG TGT TCT TTG GAC ACG AGT TTT CCC TGG AGA TCG CTT TCT GCA GGC CTA TTG GTC CTG ACT GTG GCT TCT TTT CAG-3′ (SEQ ID NO:5), respectively (see also FIG. 1C);

(xvi) exon 8, having cDNA coordinates from 1485-1573, comprising residues 603-691 of the nucleic acid sequence set forth in GenBank Accession No. L78132 and SEQ ID NO:3;

(xvii) intron 8, as comprised in the genomic sequence between the 3′ border of exon 8 (5′-GCCAAAAG-3′) and the 5′ border of exon 9 (5′-CTTTAA-3′);

(xviii) exon 9, having cDNA coordinates from 1574-1749, comprising residues 692-857 of the nucleic acid sequence set forth in GenBank Accession No. L78132 and SEQ ID NO:3;

(xix) intron 9, as comprised in the genomic sequence between the 3′ border of exon 9 (5′-ACTTTGAG-3′) and the 5′ border of exon 10 (5′-ATGATA-3′); and

(xx) exon 10, having cDNA coordinates from 1750-6101, comprising residues 858-3841, of which 858-1004 encode protein, of the nucleic acid sequence set forth in GenBank Accession No. L78132 and SEQ ID NO:3.

The present invention encompasses nucleic acid molecules spanning the region set forth in FIG. 1 or portions thereof, including nucleic acid molecules comprised solely of intronic sequence, or comprised solely of exonic sequence, or comprising both intronic and exonic sequences. Preferably such molecules are between 10 and 6200 nucleotides in length, including, but not limited to, molecules which are between 10 and 100 nucleotides in length, between 100 and 500 nucleotides in length, and between 500 and 4000 nucleotides in length.

The present invention further provides for nucleic acid molecules which hybridize to the foregoing molecules, e.g. for use as PCTA-1 encoding molecules or as probes or for antisense or ribozyme purposes, under stringent hybridization conditions, e.g., hybridization in 0.5 M NaHPO₄, 7 percent sodium dodecyl sulfate (“SDS”), 1 mM ethylenediarnine tetraacetic acid (“EDTA!”) at 65° C., and washing in 0.1×SSC/0.1 percent SDS at 68° C. (Ausubel et al., 1989, Current Protocols in Molecular Biology, Vol. I, Green Publishing Associates, Inc., and John Wiley & Sons, Inc. New York, at p. 2.10.3), and having the range of sizes set forth above.

A number of sequences have been reported for the galectin-8 gene, its encoded protein, and variants thereof that may, in specific non-limiting embodiments, be used according to the invention. These include sequences having the following GenBank Accession Nos: NT_{—}004836; XM_{—}054341; XM_{—}031635; XM_{—}031636; XM_{—}031634; XM031633; XM_{—}031632; XM_{—}031631; XM_{—}002045; NM_{—}018886; NM_{—}006499; AY 037304; AF342816; AF342815; BG231504; BG057369; BF432393; BF319030; AL136105; BE466988; BB016844; AW989355; BB015238; AW782523; AW743233; AR070783; AR070782; AR070781; AR070780; AR070779; AR070778; AF218069; AH008815; AF193806; AF193805; AW213228; AW044797; A1886585; AF074002; AF074001; AF074000; AI861917; AI819793; AI800248; AI697322; AI651369; AI647417; AI595603; AI591906; AI572630; AI429078; AI386468; AI377938; AI326142; AI220011; AI082788; AI041298; AI004574; AA918207; AA927860; AA911853; AA885888; X91790; W85929; W85928; L78132; W86887; and U09824.

The specific sequences disclosed herein may be used to identify further molecules, which may be used according to the invention, by standard techniques such as hybridization or by primer extension or PCR-based techniques. For example, a nucleic acid comprising intron 7 may be obtained using PCR primers having sequences set forth herein as being located in exons 7 and 8.

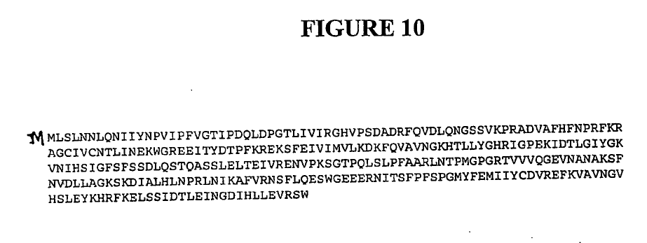

In particular embodiments, the present invention provides for “B-PCTA-1 nucleic acids”, which encode “B-PCTA-1 proteins”, defined below as having a functional first and second CRD. In specific non-limiting embodiments, where a nucleic acid molecule is to be used to produce a B-PCTA-1, the nucleic acid may have the sequence set forth in GenBank Accession No. L78132 and SEQ ID NO:3 from residues 54-1004, or another nucleic acid sequence which encodes a protein sequence, as set forth in FIG. 10 and SEQ ID NO:6 herein. The present invention further provides for nucleic acid molecules which hybridize to such sequences under stringent conditions.

For such expression purposes, the B-PCTA-1 nucleic acid may be engineered such that it is in an “expressible form”. An “expressible form” is one which contains the necessary elements for transcription and/or translation. For example, the B-PCTA-1 nucleic acid may be operatively linked to a suitable promoter element, and may comprise transcription initiation and termination sites, nucleic acid encoding a nuclear localization sequence, ribosome binding sites, polyadenylation sites, mRNA stabilizing sequences, etc.

For example, where B-PCTA-1 nucleic acid is to be transcribed into RNA, the nucleic acid may be operatively linked to a suitable promoter element, for example, but not limited to, the cytomegalovirus immediate early promoter, the Rous sarcoma virus long terminal repeat promoter, the human elongation factor-1α promoter, the human ubiquitin c promoter, etc. It may be desirable, in certain embodiments of the invention, to use an inducible promoter. Non-limiting examples of inducible promoters include the murine mammary tumor virus promoter (inducible with dexamethasone); commercially available tetracycline-responsive or ecdysone-inducible promoters, etc. In specific non-limiting embodiments of the invention, the promoter may be selectively active in cancer cells; one example of such a promoter is the PEG-3 promoter, as described in International Patent Application No. PCT/US99/07199, Publication No. WO 99/49898 (published in English on Oct. 7, 1999); other non-limiting examples include the prostate specific antigen gene promoter (O'Keefe et al., 2000, Prostate 45:149-157), the kallikrein 2 gene promoter (Xie et al., 2001, Human Gene Ther. 12:549-561), the human alpha-fetoprotein gene promoter (Ido et al., 1995, Cancer Res. 55:3105-3109), the c-erbB-2 gene promoter (Takakuwa et al., 1997, Jpn. J. Cancer Res. 88:166-175), the human carcinoembryonic antigen gene promoter (Lan et al., 1996, Gastroenterol. 111:1241-1251), the gastrin-releasing peptide gene promoter (Inase et al., 2000, Int. J. Cancer 85:716-719). The human telomerase reverse transcriptase gene promoter (Pan and Koenman, 1999, Med. Hypotheses 53:130-135), the hexokinase II gene promoter (Katabi et al., 1999, Human Gene Ther. 10:155-164), the L-plastin gene promoter (Peng et al., 2001, Cancer Res. 61:4405-4413), the neuron-specific enolase gene promoter (Tanaka et al., 2001, Anticancer Res. 21:291-294), the midkine gene promoter (Adachi et al., 2000, Cancer Res. 60:4305-4310), the human mucin gene MUC1 promoter (Stackhouse et al., 1999, Cancer Gene Ther. 6:209-219), and the human mucin gene MUC4 promoter (GenBank Accession No. AF241535), which is particularly active in pancreatic cancer cells (Perrais et al., 2001, published on Jun. 19, 2001 by J Biol. Chem., “JBC Papers in Press” as Manuscript M104204200).

Suitable expression vectors include virus-based vectors and non-virus based DNA or RNA delivery systems. Examples of appropriate virus-based gene transfer vectors include, but are not limited to, those derived from retroviruses, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6(2):113-138; Connelly, 1999, Curr. Opin. Mol. Ther. 1(5):565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculovinises, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus-or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy.

Non-limiting examples of non-virus-based delivery systems which may be used according to the invention include, but are not limited to, so-called naked nucleic acids (Wolff et al., 1990, Science 247:1465-1468), nucleic acids encapsulated in liposomes (Nicolau et al., 1987, Methods in Enzymology 1987:157-176), nucleic acid/lipid complexes (Legendre and Szoka, 1992, Pharmaceutical Research 9:1235-1242), and nucleic acid/protein complexes (Wu and Wu, 1991, Biother. 3:87-95).

B-PCTA-1 may also be produced using nucleic acid contained in plasmids, such as pCEP4 (Invitrogen, San Diego, Calif.), pMAMneo (Clontech, Palo Alto, Calif.; see below), pcDNA3.1 (Invitrogen, San Diego, Calif.), etc. Vectors useful in expressing B-PCTA in bacterial systems include but are not limited to the GST vector (Amersham) and the chitin binding domain vector (TYB-12) (New England Biolabs).

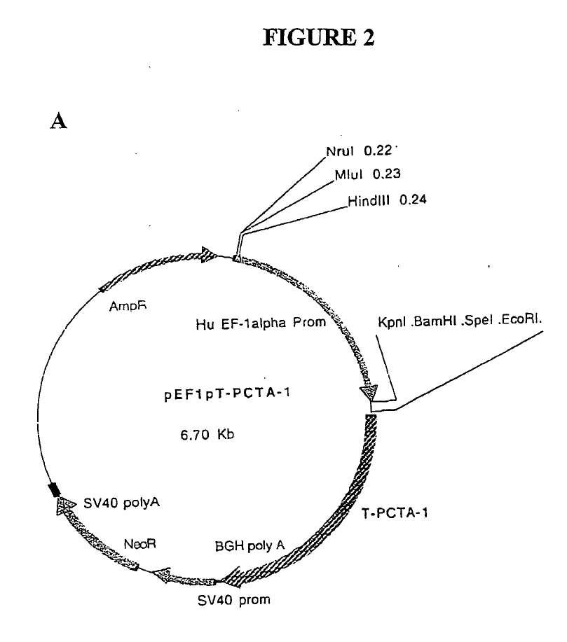

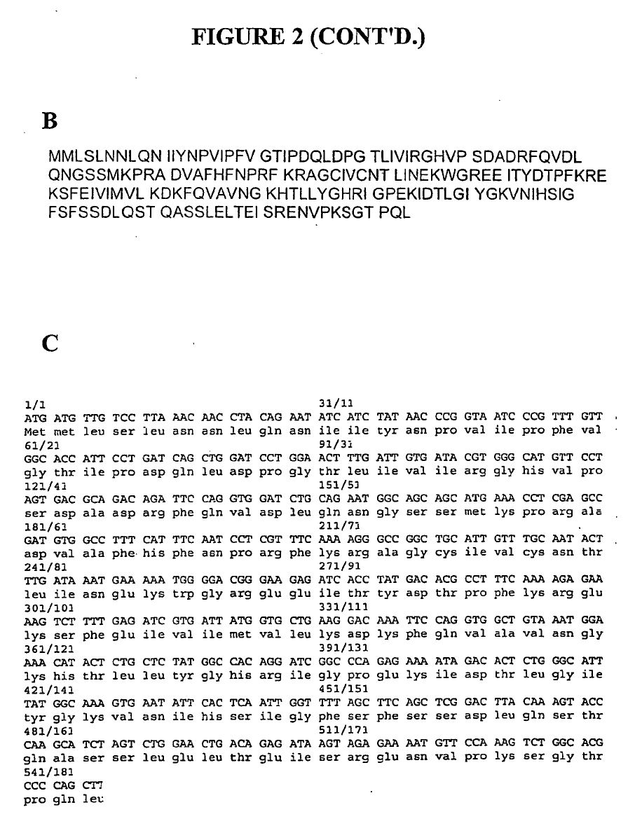

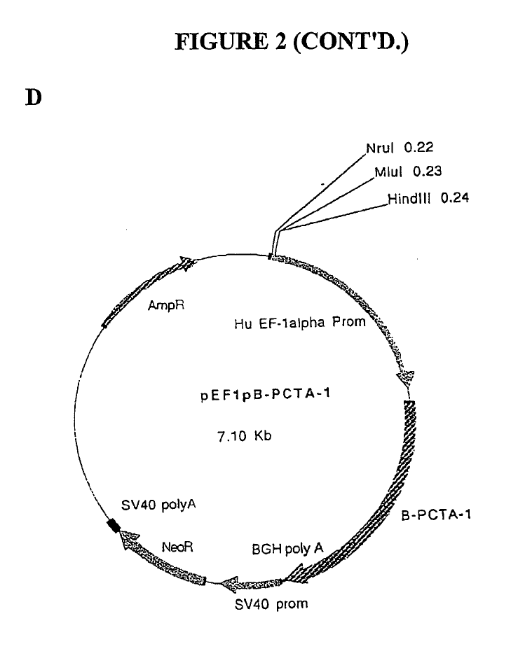

Expression systems which may be used include prokaryotic and eukaryotic expression systems, including eukaryotic cells, bacteria, fungi (e.g. yeast), insects, etc. An example of a construct which may be used to produce a B-PCTA-1 protein is shown in FIG. 2D.

Depending on the expression system used, nucleic acid may be introduced by any standard technique, including transfection, transduction, electroporation, bioballistics, microinjection, etc.

Certain embodiments of the invention, as discussed below, use nucleic acid molecules which do not encode B-PCTA-1, but rather encode a truncated PCTA-1 (“T-PCTA-1”) protein and/or have a distorted reading frame. Such nucleic acids comprise, for example, but not by way of limitation, nucleic acids which do not encode the complete second CRD, as depicted in FIG. 1A. Specific non-limiting examples include nucleic acids which comprise exons 1-7 or 2-7, but not exon 8, 9, and/or 10 (e.g., exons 1-7, exons 2-7, exons 1-8, exons 2-8, exons 1-9, exon exons 1-7, 9 and 10; exons 2-7, 9 and 10, exons 1-8 and 10, and exons 2-8 and 10). Such nucleic acids may be comprised in vectors, and may be in expressible form. One preferred specific non-limiting example of a construct which may be used to produce T-PCTA-1 is depicted in FIG. 2A.

In various embodiments, the present invention provides for oligonucleotides, antisense molecules, ribozymes as discussed below, comprising PCTA-1 nucleic acid molecules as described in this section.

In other various embodiments, the invention provides for transgenic mice comprising PCTA-1 nucleic acid molecules as described below in this section.

### 5.2 PCTA-1 Proteins and Antibodies

The present invention relates to B-PCTA-1 as well as T-PCTA-1 proteins (collectively referred to as “PCTA-1 proteins”). A “B-PCTA-1 protein” is a protein encoded by a PCTA-1 nucleic acid, as defined above, and which comprises two functional CRD domains. The CRD domains may differ from those depicted in FIG. 1 by deletion, insertion, or substitution, but they retain the ability to bind carbohydrate with at least 50 percent of the affinity of the parent molecule.

In specific, non-limiting embodiments of the invention, a B-PCTA-1 protein may have the sequence set forth in FIG. 10 (SEQ ID NO:6), or such sequence altered by conservative substitution of amino acids, preferably where the number of conservative substitutions does not exceed 10 percent of the total number of amino acids in the protein. In an alternative non-limiting, less-preferred, specific embodiment, the initial methionine residue may be omitted. A conservative substitution is one which substitutes one amino acid for another amino acid in the same class, where the classes include neutral non-polar amino acids such as glycine, alanine, valine, isoleucine, leucine, phenylalanine, proline, and methionine as well as neutral non-polar amino acid derivatives; neutral polar amino acids such as serine, threonine, tyrosine, tryptophan, asparagine, glutamine, and cysteine as well as neutral polar amino acid derivatives; acidic amino acids such as aspartic acid and glutamic acid as well as acidic amino acid derivatives; and basic amino acids such as lysine, arginine and histidine as well as basic amino acid derivatives.

In alternative embodiments, the present invention provides for T-PCTA-1 proteins which do not comprise two functional CRDs. Such truncated proteins are encoded by PCTA-1 nucleic acids, as defined in the preceding section. Amino acid sequences corresponding to the various exons are depicted in FIG. 1D. In truncated forms of the protein, all or portions of exons may be omitted relative to the full-length protein. The amino acid sequence approximately corresponding to the second CRD, as it occurs in the full-length protein and which is rendered absent or non-functional in T-PCTA-1 proteins, is set forth in FIG. 1E, SEQ ID NO: 7. In a specific, non-limiting embodiment, the present invention provides for a T-PCTA-1 protein having a sequence as depicted in FIGS. 2B and 2C, SEQ ID NO:8.

The present invention also provides for peptides representing portions of the full-length PCTA-1 molecule. Such peptides may, for example and not by way of limitation, may be peptide fragments of a particular region of the PCTA-1 molecule, for example, may be comprised in the first CRD, or in the second CRD, or in one of the exons expressed as protein, for example, as set forth in FIG. 1D (SEQ ID NOS: 9-17).

The proteins and peptides of the invention may be prepared by standard techniques, including recombinant DNA-related techniques and chemical synthesis, or by collection from natural sources. For recombinant DNA expression, vectors as set forth in the preceding section may be used.

The present invention also provides for antibody molecules which react with PCTA-1 proteins and peptides. In specific, non-limiting examples, the invention provides for antibody molecules (as defined below) which bind specifically to proteins having a sequence as set forth in SEQ ID NO: 6 (a species of B-PCTA-1), SEQ ID NO: 7 (the second CRD, as shown in FIG. 1E), SEQ ID NO: 8 (a species of T-PCTA-1, as shown in FIG. 2B) and/or to a peptide having an amino acid sequence as set forth in SEQ ID NO: 9, 10, 11, 12, 13, 14, 15, 16 or 17 (shown in FIG. 1D). Antibodies of varying specificity may be used, for example, to distinguish between B-PCTA-1 and T-PCTA-1 proteins, or to determine the presence or absence of one or both CRDs in a PCTA-1 protein. The ability to make such a distinction may be useful, for example, for diagnostic or therapeutic purposes.

According to the invention, a PCTA-1 protein or peptide, derivatives (e.g. histidine tagged protein), or analogs thereof, may be used as an immunogen to generate antibodies. Such antibodies include, but are not limited to, polyclonal, monoclonal, chimeric, single chain, Fab fragments, and a Fab expression library.

Various procedures known in the art may be used for the production of polyclonal antibodies which specifically bind to a B-PCTA-1 or T-PCTA-1 protein or peptide. For the production of antibody, various host animals can be immunized by injection with the protein or peptide, including but not limited to rabbits, mice, rats, goats, etc. Various adjuvants may be used to increase the immunological response, depending on the host species, and including but not limited to Freund's (complete or incomplete) adjuvant, mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corptebacteriumparvum.

For preparation of monoclonal antibodies directed toward one of the foregoing PCTA-1 proteins or peptides, any technique which provides for the production of antibody molecules by continuous cell lines in culture may be used. Examples of such techniques include the hybridoma technique originally developed by Kohler and Milstein (1975, Nature 256:495-497), as well as the trioma technique, the human B-cell hybridoma technique (Kozbor et al., 1983, Immunology Today 4:72), and the EBV hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). Further, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for a PCTA-1 protein or peptide together with genes from a human antibody molecule of appropriate biological activity may be used; such antibodies are within the scope of this invention.

According to the invention, techniques described for the production of single chain antibodies (U.S. Pat. No. 4,946,778) may be adapted to produce PCTA-1 protein or peptide-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity.

Antibody fragments which contain the idiotype of the molecule can be generated by known techniques. For example, such fragments include but are not limited to: the F(ab′)₂, fragment which can be produced by pepsin digestion of the antibody molecule; the Fab′ fragments which can be generated by reducing the disulfide bridges of the F(ab′)₂, fragment, the Fab fragments which can be generated by treating the antibody molecule with papain and a reducing agent.

### 5.3 Methods of Inhibiting Cancer Cell Proliferation and/or Metastasis

The present invention provides for methods of inhibiting the proliferation, anchorage independent growth, and/or metastasis of a cancer cell by introducing, into the cancer cell a B-PCTA-1 nucleic acid, or by otherwise increasing the intracellular levels of a B-PCTA-1 protein. Alternatively or in addition, the method may include the step of exposing the cancer cell to extracellular B-PCTA-1 protein which contacts the cancer cell surface.

The cancer cell may be selected from the group of cancer cells including, but not limited to, prostate cancer cells, cervical cancer cells, lung cancer cells, breast cancer cells, melanoma cells, colon cancer cells, bladder cancer cells, leukemic cells, lymphoma cells, hepatocellular carcinoma cells, pancreatic cancer cells, gastric cancer cells, renal cancer cells, thyroid cancer cells, and central nervous system cancer cells.

B-PCTA-1 nucleic acids in expressible form as set forth in section 5.1 above may be used. In specific non-limiting embodiments, a B-PCTA-1 nucleic acid in expressible form may be comprised in a vector selected from the group consisting of an adenovirus vector, an adeno-associated virus vector, and a retrovirus vector. Such a viral vector may be then be used to infect the cell. In preferred embodiments, a replication defective adenovirus vector may be used. The vector containing the B-PCTA-1 nucleic acid may be comprised in a suitable pharmaceutical carrier and administered by a suitable route, including, but not limited to, intravenous, intrathecal, intraperitoneal, intraarterial, subcutaneous, intramuscular, etc., and/or may be directly administered into an affected tissue and/or into a tumor.

Alternatively, a B-PCTA-1 protein may be administered to the cancer cell. To improve the stability of the protein, it may be incorporated into a microparticle, liposome, or other protein-stabilizing formulation. Further or in the alternative, B-PCTA-1 protein may be instilled directly into a tumor site.

The effectiveness of the foregoing methods on a particular species of cancer cell may be tested by determining whether the introduction of and/or exposure to PCTA-1 suppresses transformation-associated characteristics of the cell. Such characteristics include cellular morphology, proliferation rate, the ability for anchorage-independent growth, lack of contact inhibition, increased expression of tumor-associated antigens, decreased expression of differentiation associated antigens, and tumorigenicity in vivo in animal models, where suppression or increase is preferably by at least about 25 percent relative to control values. In a preferred non-limiting embodiment, the effectiveness of B-PCTA-1 in suppressing the transformed phenotype may be measured as an inhibition of colony formation in soft agar, as described in the example sections below.

The foregoing methods may be used in the treatment of a subject suffering from a cancer, comprising administering, to the subject, a therapeutically effective amount of B-PCTA-1 nucleic acid or protein. A therapeutically effective amount of these agents produces one or more of the following results: a decrease in tumor mass, a decrease in cancer cell number, a decrease in serum tumor marker, a decrease in tumor metastasis, a decreased rate of tumor growth, improved clinical symptoms, and/or increased patient survival. The cancer may be first treated surgically to de-bulk the tumor mass, if appropriate. In specific non-limiting embodiments, the cancer from which the subject is suffering may be prostate cancer, cervical cancer, lung cancer, breast cancer, melanoma, colon cancer, bladder cancer, leukemia, lymphoma, hepatocellular carcinoma, pancreatic cancer, gastric cancer, renal cancer, thyroid cancer, or central nervous system cancer.

As shown in the example sections below, a specific effect of B-PCTA-1 is its ability to inhibit growth of tumor cells in soft agar. Cifone and Fidler, 1980, Proc. Natl. Acad. Sci. U.S.A. 77:1039-1043 showed that anchorage independent growth of tumor cells in Noble agar semisolid medium is selective and permits the isolation of metastatic subpopulations in cells. Accordingly, the discovery that B-PCTA-1 selectively inhibited growth of cells in soft agar (as compared to monolayer cultures) indicates that the foregoing methods may be particularly useful in the treatment of subjects suffering from metastatic cancer or in a cancer having a tendency to metastasize.

These methods may be used alone or in combination with other forms of therapy, including, but not limited to, chemotherapy, surgery, immunotherapy, and/or radiation therapy. As shown in example section 7, below, the inhibitory effect of B-PCTA-1 expression was enhanced by co-exposure of cancer cells to beta interferon. Accordingly, in specific non-limiting embodiments of the invention, the foregoing methods may be practiced in conjunction with the administration of a differentiation-promoting agent or cytoline, such as, but not limited to, beta-interferon, mezerein, histone deacetylase inhibitors, retinoic acid, Vitamin D, butyric acid, and phenylacetate.

### 5.4 Methods of Inhibiting the Expression of an Oncogenic PCTA-1 Protein

Another aspect of the present invention involves the discovery that T-PCTA-1 expression promotes oncogenic characteristics. Accordingly, the invention provides for methods of reversing the malignant process in a cancer cell expressing T-PCTA-1 which comprise exposing the cell to an agent which inhibits the expression or action of the truncated protein.

The expression, by a cancer cell, of a T-PCTA-1 protein may be determined using standard techniques. For example, the cancer cell may be determined, at the RNA level, to express a splice variant or another natural variation which would result in a T-PCTA-1 protein. At the protein level, total cellular protein could be collected and analyzed to determine whether a T-PCTA-1 protein is present, for example, on a Western blot using an antibody that reacts with PCTA-1 protein (e.g., an anti-PCTA-1 antibody as described above or an anti-galectin 8 antibody which binds to PCTA-1). As another non-limiting example, immunohistochemical techniques could be employed to determine the presence of a T-PCTA-1 in a cell section or at the cell surface, in which the level of binding of an antibody specific to the second CRD of PCTA-1 could be determined (and optionally compared to binding of an antibody specific to the first CRD of the protein).

In a cell which is expressing a T-PCTA-1 protein, it may be desirable to inhibit all PCTA-1 expression, whether it involves only expression of the truncated protein or of full-length PCTA-1 as well, in view of the oncogenic properties of the truncated protein. Expression may be inhibited by any method known in the art, including, but not limited to, the use of antisense molecules directed to all or a portion of the PCTA-1 coding region. Such antisense molecules may be administered as oligonucleotides (for example, but not by way of limitation, phosphorothioate-linked oligonucleotides) or via a vector such as a virus (for example, but not by way of limitation, a replication-defective adenovirus).

Alternatively, if it can be shown that truncated protein is a result of the expression of a particular splice variant of a B-PCTA-1 RNA transcript, it may be desirable to target such variants for specific destruction. Such specific destruction may be provided through the use of ribozyme molecules which selectively bind to a region of mRNA present in the splice variant but not present in a B-PCTA-1 encoding transcript (Rossi, 1994, Current Biology 4:469-471; U.S. Pat. No. 5,093,246 by Cech et al., issued Mar. 3, 1992; Haseloff and Gerlach, 1988, Nature 334:585-591; Zaug et al., 1984, Science 224:574-578; Zaug and Cech, 1986, Science 231:470475; Zaug et al., 1986, 324:429433; Been and Cech, 1986, Cell 47:207-216).

Further, the invention provides for methods of selectively destroying a cancer cell that expresses a T-PCTA-1, comprising exposing the cancer cell to an effective amount of an antibody that binds to T-PCTA-1 but does not significantly bind to B-PCTA-1, such that antibody binding results in destruction of the cancer cell. Such destruction may be accomplished, for example, by immunologic means (e.g., via antibody-dependent cellular cytotoxicity) or by a toxin or other bioactive agent linked to the antibody molecule. An antibody may selectively bind to a truncated version of PCTA-1 where the truncation results in an alteration of the secondary or tertiary structure of the protein, creating new epitopes.

### 5.5 Methods of Diagnosing Malignancy

The present invention further provides for methods of identifying a malignant cell and therefore diagnosing malignancy in a subject comprising detecting, in a cell of the subject, a T-PCTA-1 protein. Such detection may be accomplished as discussed above, using, for example, a Western blot that reveals a PCTA-1 protein having a reduced molecular weight or an antibody that selectively binds to truncated PCTA-1. The presence of a T-PCTA-1 protein has a positive correlation with the presence of a malignancy.

### 5.6 Preparation of Model Systems

The present invention provides for the preparation of model systems for malignancy comprising introducing, into a cell, a nucleic acid encoding a T-PCTA-1 gene in expressible form and then selecting for a transformed characteristic, e.g., the ability to grow in soft agar. Such transformed cells may be useful for studying the mechanisms involved in oncogenesis and tumor spread, and may be useful for identifying agents useful in treating cancer and/or preventing metastasis. For example, small molecules could be screened for their ability to reverse the changes resulting from introduction of a T-PCTA-1, e.g., the ability to inhibit colony formation in soft agar.

In analogous systems, a nucleic acid encoding a B-PCTA-1 gene, in expressible form, may be introduced into cells and then those cells may be used to screen for agents, such as small molecules, which augment the anti-transformation effects of B-PCTA-1 (e.g., the ability to inhibit colony formation in soft agar).

In further embodiments, the present invention provides for transgenic animals containing transgenes which encode B-PCTA-1 or T-PCTA-1 proteins. For example, a transgenic mouse may be prepared carrying a transgene encoding the full-length B-PCTA-1 protein (e.g., a protein having the amino acid sequence as shown in FIG. 10) under the control of the elongation factor-1α promoter or other promoters active in cells of the prostate gland, among other sites. Such animals may be singly transgenic, expressing the B-PCTA-1, or related proteins such as the truncated form of B-PCTA-1 (T-PCTA-1) or any homologues possessing similar biological functions, in various cells and tissues of otherwise phenotypically normal (i.e. wild-type) mice, including the epithelial cells of the prostate gland, or doubly transgenic, in which expression of the B-PCTA-1 or these same related proteins may occur in cell or tissues of the animal that further comprise deletions, insertions or other alterations of the genome. Such deletions, insertions or alterations of the genome may be those that cause or influence the development or spread of prostate cancer. In a preferred embodiment, the transgenic mice comprise the human B-PCTA-1 gene under the transcriptional control of the human elongation factor-1α promoter. In a more preferred embodiment, the transgenic mice are doubly transgenic, and comprise both the human B-PCTA-1 gene under the transcriptional control of the human elongation factor-1α promoter and the SV40 T antigen gene under the transcriptional control of the prostate-specific rat probasin promoter.

In light of the ability of B-PCTA-1 expression to reduce the growth of transformed cells when cultured in soft agar, singly transgenic B-PCTA-1 mice may be useful as models to determine the role of B-PCTA-1 on the processes of cell proliferation, cell migration and development. These animals may also be used to identify and characterize agents that modulate the effects of PCTA-1. Such agents may be useful adjuncts in the treatment of certain proliferative diseases, including cancers.

Similarly, the doubly transgenic B-PCTA-1/TRAMP animals also may be useful as models to determine the role of B-PCTA-1 on the processes of cell proliferation, cell migration and development. The apparent suppression of prostate tumor development observed in these animals suggests that B-PCTA-1 expression in vivo et situ can suppress tumor formation. Thus, further characterization of these animals may provide insights into the tumorigenic process. The animals may also be employed to identify agents that enhance the tumor suppressive effects, leading to the development of new therapeutic modalities for the management of cancers of the prostate. Insights gained from these studies may also be relevant to other tumor types in which autochthonous mouse models exist.

### 6. EXAMPLE

### Characterization of the PCTA-1 Gene and the Effect of Overexpression of Full-Length PCTA-1 on Tumor Cells

### 6.1 Materials and Methods

Cell lines, tissue culture and transfection. The LNCAP, PC-3 and DU145 prostate cancer cell lines were obtained from the ATCC and maintained according to the instructions provided. The HO-1 and C8161 melanoma cell lines were cultured and maintained as previously described (Jiang et al., 1996, Proc. Natl. Acad. Sci. U.S.A. 93: 9160-9165; Su et al., 1998, Proc. Natl. Acad. Sci. U.S.A. 95: 14400-14405; Welch et al., 1994, Oncogene 9: 255-262). Transfection of cells was performed using the Superfect reagent (Qiagen) according to the supplier's instructions.

Genomic and cDNA clone analyses. A human genomic BAC library (Genome Systems Release II) was screened using a ³²P-labeled PCTA-1 ORF probe. Analysis of the isolated clones was performed by subcloning fragments in plasmid vectors and sequence analysis using plasmid or cDNA derived primers. Southern blotting and PCR analyses was used to map and order the different regions of the gene. Sequence analysis was performed using the Fasta, Gap and Bestfit subroutines in the GCG sequence analysis package (Genetics Computer Group Inc) and BLAST searches at the NCBI.

Transcriptional analyses. Total RNA was prepared using the RNAeasy kit (Qiagen) according to the supplied protocols and hybridization was performed using UltraHyb (Ambion) and the EasyStrip (Ambion) systems when the same blot was to be reprobed. Northern blot analysis was performed as described previously (Gopalkrishnan et al., 1999, Nucleic Acids Res. 27: 4775-4782) and washing conditions were stringent (0.2×SSC/55-60° C.) to prevent cross hybridization of related species of RNA. RT-PCR analyses was performed by reverse transcribing 1 μg of total cellular RNA using Superscript II (Life Technologies Inc.) at 42° C. for 90 min in the supplied buffer, followed by one or two rounds of PCR with gene specific primers for 25-30 cycles. Primer extension analysis was performed essentially as described previously (Gopalkrishnan et al., 1996, Oncogene 13: 2671-2680), except that 50-75 μg total RNA was required to obtain signals for low abundance PCTA-1 mRNA. Commercial multiple tissue Northern blots were used as recommended by the manufacturer (Clontech) except that the EasyStrip system (Ambion) was used to prepare probes to enable multiple use of each blot.

Western blotting and immunofluorescence. Cells transfected with empty vector or GFP/PCTA-1 cells were harvested on ice with RIPA buffer containing a protease inhibitor mix (Roche), spun at 15,000 g for 30 min at 4° C. and supernatant loaded in 1× Laemelli dye on a 10% SDS -PAGE, transferred by electroblotting onto nitrocellulose membranes and probed with anti-GFP monoclonal antibody (Clontech). Cells were transfected with appropriate vectors after plating overnight on sterilized cover slips. Cells were washed three times in PBS and directly mounted in PBS on a coverslip to observe expression and localization of PCTA-1 under a fluorescence microscope (Nikon).

Stable cell lines, soft agar colony formation assays, adenovirus transduced expression. Stable cell lines were constructed using the pCDEF3 expression vector (Gopalkrishnan et al., 1999, Nucleic Acids Res. 27: 4775-4782) and selecting clones with G418 followed by screening for expression by Northern blotting. Agar colony formation assays with stable expressing cell lines or adenovirus infected cells were performed essentially as described in (Jiang et al., 1996, Proc. Natl. Acad. Sci. U.S.A. 93: 9160-9165; Su et al., 1998, Proc. Natl. Acad. Sci. U.S.A. 95: 14400-14405; Welch et al., 1994, Oncogene 9: 255-262).

### 6.2 Results

The PCTA-1 genomic locus. PCTA-1 maps to the chromosome region 1q42-43, a locus which has been identified in a recent European study (Berthon et al., 1998, Am. J. Hum. Genet. 62: 1416-1424) to contain a predisposing gene for early-onset prostate cancer based on analyses of several families with a recorded genetic predisposition for the disease. Screening of a bacterial artificial chromosome human genomic library (Genome Systems Inc.) yielded two overlapping clones that spanned the entire PCTA-1 locus. Clones were sequenced to determine the exon/intron junctions using appropriate primers and alignment to the cDNA sequence. The results of this analysis (FIG. 1A) showed that the locus was comprised of eight constitutively present exons (exons 2-9), having exonic boundaries as set forth in FIG. 1B. Two additional exons (7′ and 7″) (FIGS. 1A and 1C) are represented only in a sub-population of PCTA-1 message due to alternative splicing. The two exons at the extremities (exons 1 and 10) are present as partial or complete entities in the mature message due to internal processing.

The 5′ most exon is present in the mature transcript either as a single 828 nt unit or is processed internally (FIG. 2A) to give a 488 nt product. We were initially unaware of the smaller processed transcript since our RT-PCR analyses with prostate cancer cell RNA only detected the larger product. Primer extension analysis (FIG. 2B) using a primer close to the 3′ extremity of the exon yielded two extension products implying the possibility of two transcription start sites. Sequence analysis of the exon and a database search determined that transcription initiates at a single site (FIG. 2A, +1 “C” residue) and the two products represent the non-truncated or additionally processed forms of the first exon. There is a TATA-box consensus sequence present 43 bp upstream of the identified transcription start site indicating that the promoter falls into the TATA-containing class. Database searches identified three sequences with 5′ regions identical to the shorter processed form of exon 1 (Accession numbers AF074000, AF074001 and AF074002). These entries correspond to differentially processed forms of a cDNA closely related to PCTA-1, designated Po66 and isolated from human lung squamous carcinoma (Bassen et al., 1999, Anticancer Res. 19: 5429-5433). It is possible that the predominant mRNA species vary depending on the tissue source of isolation, though we can not rule out the possibility that our conditions for RT-PCR caused a bias toward detection of the larger isoform since both are clearly detected in primer extension analysis using prostate derived RNA.

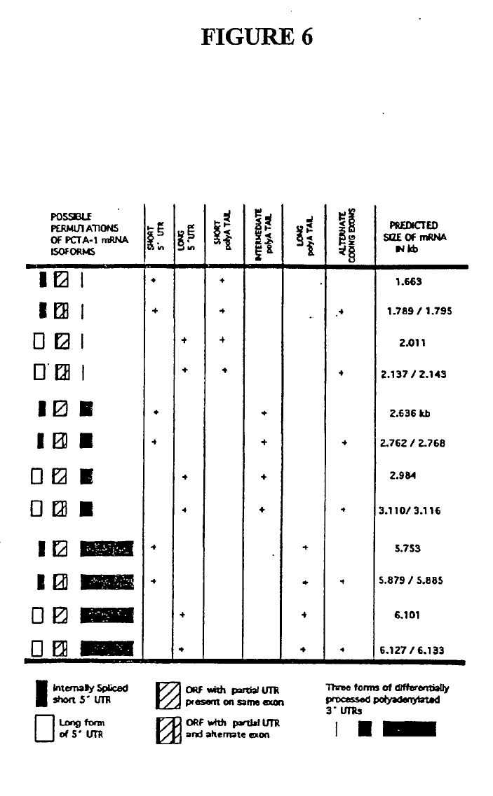

There is an overall conservation of structure for the exons encoding the carbohydrate binding domains of PCTA-1 (FIG. 1A) compared to that observed for the other galectins. In general, genomic loci encoding galectin CRDs contain a 3-exon unit implying a common evolutionary origin (Gitt et al., 1998, J. Biol. Chem. 273: 2961-2970; Hadari et al., 1995, J. Biol. Chem. 270: 3447-3453). In the tandem repeat galectins, each of two separate CRDs are encoded by two such 3-exon units separated by exons for the intervening linker region, an architecture seemingly conserved in the PCTA-1 locus (FIG. 1A, exons 3-5 for CRD-1 and 8-10 for CRD-2). The linker region in Galectin-6 is spread over two exons as initially seemed to be the case in PCTA-1. However, based on alignment of sequences reported in AP074001 and AF074002 with our BAC derived genomic sequence, two additional exons (FIG. 1A, exons 7′ and 7″) were discovered. ESTs in current databases with identity to alternate exons 1 and 2 (FIG. 1A, 7′ and 7″, FIG. 1C and FIG. 6, ALT1 and ALT2) are present at frequency of one and four independent cDNAs respectively, compared to 42 independently reported sequences for the PCTA-1 isoform wherein these elements are absent.

It therefore seems that these alternately spliced exons (FIG. 6, ALT1 and ALT2) constitute relatively minor proportions of total PCTA-1 message, with the major form present in cells most likely containing exon 7 directly spliced to exon 8. We have therefore preferred to designate these alternatively spliced exons as 7′ and 7″. Conceptual translation of the ORF without the alternate exons or with each one individually or both linked together in tandem give rise to proteins related to PCTA-1 (i.e. the reading frame of the PCTA-1 ORF continues to be maintained) other than an extended linker region. To our knowledge this is the first instance of galectin peptide modification of this nature and is likely to impact on function and carbohydrate binding capacity since the two CRD will be spaced further apart as a result of the expanded linker region contributed by inclusion of one or both alternative exons. FIG. 1B shows the exonic part of the splice junction boundaries of all the constitutively present exons and the 5′ UTR-All but one of the exons (exon 7″) are flanked by consensus splice donor and acceptor site GT:AG sites.

The sequence of the two alternate exons that have been previously reported in AF074001 and AF074002 are shown in FIG. 1C (SEQ ID NOS: 4 and 5) Alternate exon 1 has consensus splice donor and acceptor sites and is likely to be included or excluded depending on whether it is by-passed or not by the splicing machinery. Alternate exon 2 as originally reported (AF074002) has a 5′GG instead of AG splice consensus sequence. Three additional C residues differing from those reported in AF074002 were observed in our analyses, (‘Cs’ in above sequence; confirmed by several sequencing passes over the same genomic region and also present in EST database sequences). In addition, alternate exon 2 is physically contiguous with exon 8 in genomic DNA (FIG. 1A). The last codon in this exon codes for a glutamine residue (CAG). It appears that AG-residues which are part of the last codon (CAG) of exon 7″ doubles as a 5′ splice consensus site for exon 8, to join the 3′ end of exons 7 or 7′ to exon 8. It appears that the splicing machinery recognizes the AG sequence at the 5′ end of exon 7″ positions 55 and 56), at a much lower frequency, and includes it in combination with exon 8 as a single exonic unit.

Current conceptual translation of Alternate Exon 2 with the additional C residues demonstrate that the ORF can only be maintained if splicing occurs at position 57 and not at the originally indicated sequence (AF074002). Based on our sequence analyses and existence of EST sequences in the databases, it appears that the region around this exon is error prone with respect to splicing. Many EST sequences and the sequence reported in AF074002 seem to be products of partial mRNA processing leaving behind parts of un-or incorrectly spliced intronic sequences. Based on the frequency of occurrence (in the EST database) it appears that transcripts containing these alternate exons are much less frequent than transcripts excluding them.

At the present time it is unclear as to what signals or events govern selective occurrence of one type of processing event over the other. It also appears that many different isoforms are expressed in varying amounts by a given cell population and possibly within the same cell at all times (see below). The 3′ UTR of PCTA-1 is encoded by differential processing of a single large 4352 bp exon. This exon contains (FIG. 1A, exon 10) 147 bp of coding sequence followed by the stop codon and remaining part comprising UTR sequence. The UTR is processed by the 3′ processing polyadenylation machinery to generate three differentially polyadenylated species of PCTA-1 mRNA having UTR lengths of 126, 1103 and 4217 bp respectively, when calculated from the stop codon. It is interesting to note that the polyadenylation consensus sequence AAUAAA is not present at any of the three sites of processing (putative signals for polyadenylation at the respective sites matching most closely to the consensus, based on position and sequence are TTAAAAT, TTAAAA and AATGTGAAA, respectively). The location of 3′ termini was derived from direct sequence analysis of oligo-dT primed RT-PCR products and sequence comparisons with database information.

The original 3.8 kb message reported for PCTA-1 (Su et al., 1996, Proc. Natl. Acad. Sci. U.S.A. 93: 7252-7257) appears to have been primed from a stretch of A-residues present in genomic DNA encoding the 3′ UTR and likely represents oligo-dT priming from a site that is not a genuine poly-A tail.

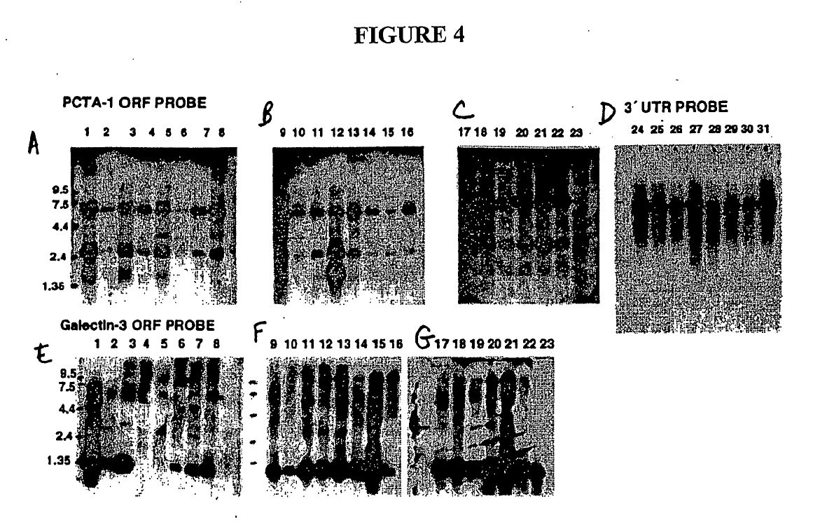

The PCTA-1 locus encodes multiple mRNA species that are differentially spliced and/or differ in the site of polyadenylation. Northern blot analyses using a probe containing the ORF of PCTA-1 detected three major species of RNA having sizes corresponding to approximately 1.6, 2.6, and 6.0 kb. These species were detected irrespective of whether total RNA or enriched polyadenylated fractions were used (FIGS. 4A-G and 5A-H). PCTA-1 displayed a ubiquitous pattern of expression in all human tissues analyzed (FIG. 4A-C, PCTA-1 ORF probe). Relatively high expression was seen in heart, placenta, liver, pancreas, spleen, testis, ovary, spinal cord, lymph node, trachea and adrenal gland. Intermediate expression was observed in lung, kidney, prostate, peripheral blood lymphocytes, stomach and thyroid. Low expression occurred in whole brain, skeletal muscle, thymus, small intestine, colon and bone marrow.

Due to partial homology at the nucleotide level between the different galectins, there was concern that use of an ORF probe (PCR product spanning ATG to stop codon) might cross hybridize to related and more highly expressed galectin RNAs. A galectin-3 ORF cDNA (PCR product spanning ATG to stop codon) was used to reprobe the multiple tissue Northern blot (FIG. 4E-G, Galectin-3 ORF probe). An anticipated signal of 1.3 kb for galectin-3 was dissimilar in size, intensity and tissue distribution from that obtained with PCTA-1, indicating that probes and hybridization conditions used in these studies distinguished between related sequences. Overall, the PCTA-1 signal and therefore message abundance seemed to be lower than Galectin-3 (the PCTA-1 blots were exposed twice as long) and this was true for several other experiments where PCTA-1 signals were only visible after relatively longer exposures compared to other genes probed on the same blot. In fact, it was not possible to detect PCTA-1 RNA by reprobing a previously used blot, pointing towards overall rareness of message abundance of PCTA-1. A PCR amplified probe corresponding to the 3′ UTR sequence beyond the poly-A site of 1.6 and 2.6 transcripts (Su et al., 1996, Proc. Natl. Acad. Sci. U.S.A. 93: 7252-7257), detected only the 6 kb product (FIG. 4D, lanes 24-31) confirming that the relationship between the transcripts detected by the ORF probe is in part due to differing 3′ UT lengths as described in the above sections and FIG. 6.

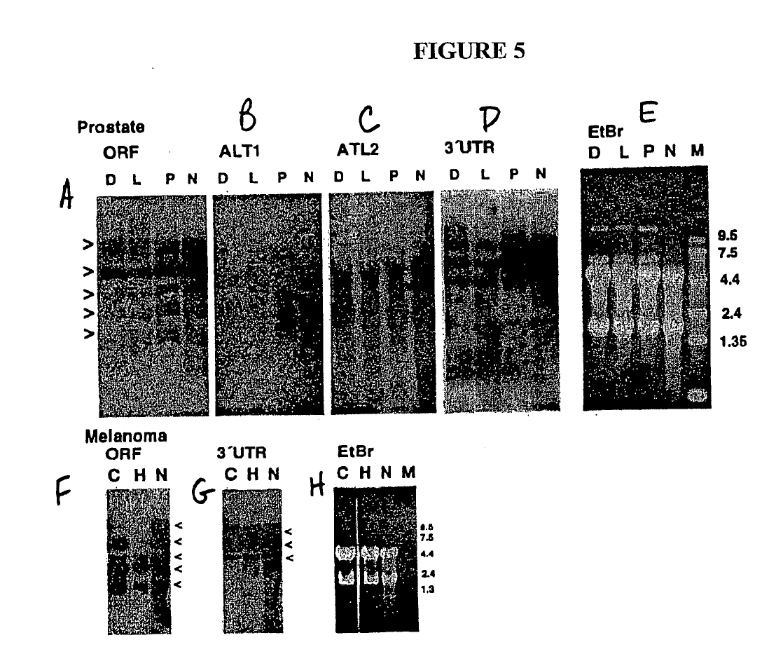

From results obtained with the Northern blots containing normal human tissue derived RNA it is clear that PCTA-1 is expressed at detectable levels in many normal tissues (FIG. 4A-G). To determine if there was any correlation between expression status and cellular transformation, Northern blot analyses were performed with prostate cancer and melanoma cell line derived RNA (FIG. 5A-H). Two aspects of PCTA-1 expression were analyzed in these series of experiments: first, to determine whether the level of PCTA-1 expression was enhanced as a consequence of transformation; and second, to observe if the presence of specific isoforms was correlated with transformed cells.

In addition to detecting the three major transcripts described above (normal tissue derived poly-A RNA), we were able to consistently observe two additional bands of around 4.5 and 9.0 kb (FIG. 5A-H, first and third arrowheads from top) with total RNA from prostate or melanoma cell lines as well as normal human prostate tissue RNA (Clontech) using the ORF probe. The origin of the 9.0 kb transcript is presently unclear and attempts to RT-PCR this sequence using oligo-dT and known 3′ UTR primers have not been successful.

After considering all the theoretically possible isoforms (FIG. 6), it is also difficult to account for a 4.5 kb transcript. These additional species might represent partially processed forms of the nascent transcript since total RNA was used in these experiments. These two forms are not detectable with polyA-RNA and further analysis is presently underway. It is apparent that the three larger species (4.5-9.0 kb) contain the 3′ UTR region extending beyond the terminus of the 2.6 kb transcript since they hybridize to a probe in that region (FIGS. 5D and 5G, 3′ UTR panels). It appears that the alternate exons (FIGS. 1A and 1C, exons 7′ and 7″) are present in at least a sub-population of PCTA-1 transcripts since positive signals were obtained using specific PCR generated probes containing only these sequences. It is possible that these alternate exons and corresponding larger forms of protein, have a higher prevalence in transformed tissues (FIGS. 5B and 5C, panels ALT1 and ATL2, compare lanes D and P to N) (Bassen et al., 1999, Anticancer Res. 19: 5429-5433). However, the low abundance of ALT1 transcripts in LNCaP prostate cancer samples argues against such a generalization.

A complete lack of consistency of PCTA-1 isoform expression has been observed in a given cell type. There appears to be a shift in the overall composition and individual abundance of a given isoform within the same cell type harvested at different times. We have attempted to determine if growth conditions influence these changes by harvesting RNA from cells grown at high, median and low cell densities but this does not have any apparent effect in causing selective expression of a given isoform. Results as they stand at the present time seem to indicate that regulation of the composition and abundance of isoforms appears to be stochastic in nature. The one reproducible pattern has been an apparent higher overall expression level of the sum total of all isoforms in cell lines that have the capacity to form metastatic tumors in nude mice, namely the PC-3 prostate cancer and C8161 melanoma cell lines, compared to expression levels in normal prostate or non-metastatic cell lines (FIG. 5A-H, compare lanes P to others in FIG. 5A-D and lanes C to others in FIGS. 5F and 5G). This phenomenon was observed in several independent batches of RNA.

The predicted number of possible permutations of different variable elements in the PCTA-1 gene transcription unit stands at 18 (FIG. 6, right hand column). Many of these isoforms may not be resolved as separate species during electrophoresis depending on gel running conditions and abundance (e.g. 1.6-1.79, 2.011-2.143, 2.7-2.9 and 5.7-6.1 kb species) while others have at the present time not been observed (2.9-3.1 kb species).

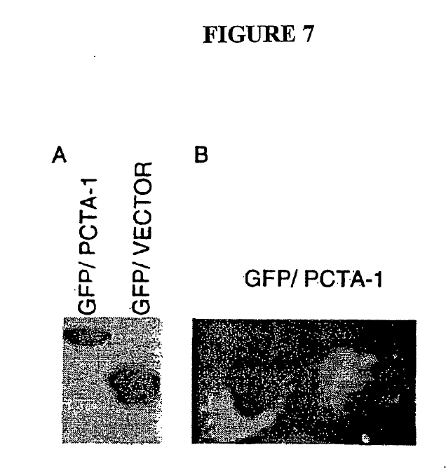

Intracellular localization and phenotypic effect of overexpression. Intracellular location can provide important clues in discerning the biological role of a protein of undetermined function. Eukaryotic plasmid expression vectors were constructed to express a GFP/PCTA-1 fusion protein. An advantage of using this type of analysis is that expression may be observed in live unfixed cells avoiding potential artifacts generated by sample fixation protocols. Expression of the correct sized GFP PCTA-1 fusion protein was determined by Western blotting of whole cell extracts from HeLa cells transiently transfected with the GFP PCTA-1 expression vector (FIG. 7A). Live unfixed samples transfected in parallel were observed for expression and intracellular localization of PCTA-1 (FIG. 7B) by fluorescence microscopy. PCTA-1 expression was extra-nuclear, expression was observed only in the cytoplasm (as compared to wild-type GPF vector control, which was evenly distributed in both compartments, data not shown). Expression was however not uniformly spread throughout the cytoplasm and showed a micro-clustering pattern reminiscent of that seen with proteins associated with mitochondria, the Golgi or trans-Golgi membranes and observed for some other galectins (Bassen et al., 1999, Anticancer Res. 19: 5429-5433; Hadj et al., 1996, J. Cell. Biochem. 62: 529-542; Lutomski et al., 1997, Glycobiology 7: 1193-1199; Maldonado et al., 1999, Invest. Ophthalmol. Vis. Sci. 40: 2971-2977; Maquoi et al., 1997, Placenta 18: 433-439; Sarafian et al., 1998, Int. J. Cancer 75: 105-111; Su et al., 1996, Proc. Natl. Acad. Sci. U.S.A. 93: 7252-7257).

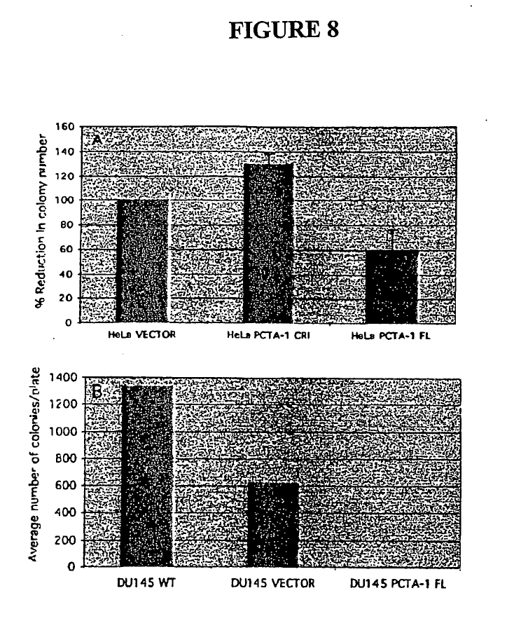

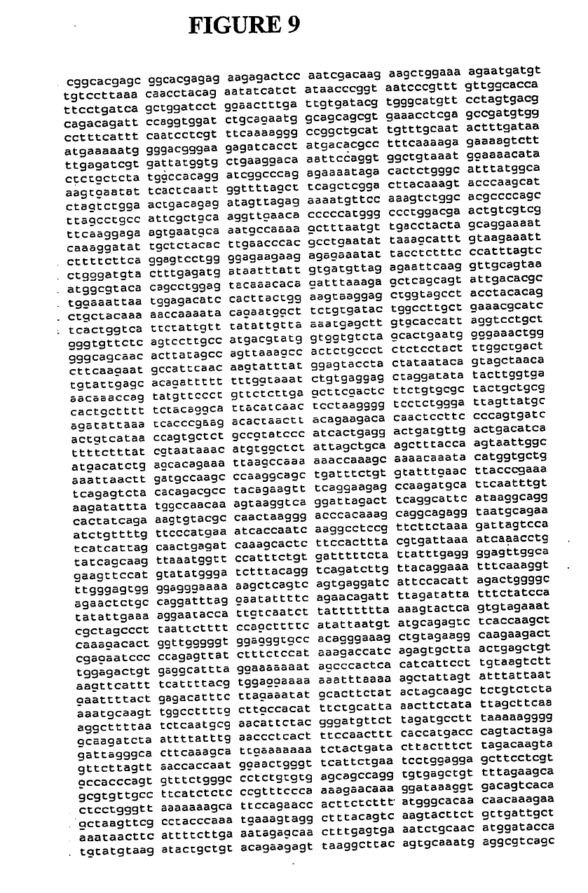

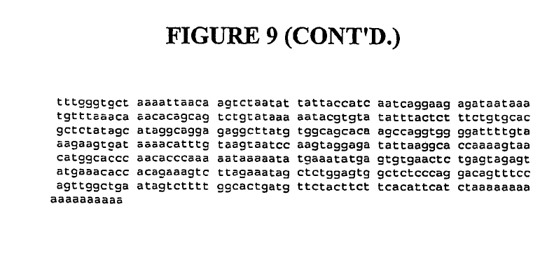

To determine the phenotypic effects of overexpression, eukaryotic expression vectors expressing the full-length ORF or a deletion of the second CRD (δCR1) were constructed, transfected into HeLa cells and stably expressing clones were isolated by drug selection. These clones were analyzed by Northern blot to confirm continued stable expression of PCTA-1 (not shown). Soft-agar colony formation assays were performed with stable cell lines expressing full-length, truncated and vector control cells. Continued stable over-expression of PCTA-1 in cells inhibited the ability to form colonies in soft agar by an average of 40% compared to HeLa vector control (FIG. 8A) while cells expressing the truncated version of PCTA-1 displayed around 30% enhanced rate of soft agar colony formation.

The ability of full length PCTA-1 to inhibit colony formation was independently confirmed by infecting DU-145 cells with a non-replicating Adenovirus vector expressing full-length PCTA-1. While the empty Adenovirus control vector inhibited colony forming ability of these cells by 50%, probably due to non-specific toxicity, no colonies were observed in cells infected with a vector expressing PCTA-1 i.e. an inhibition of 100% was observed. Adenoviruses have a close to 100% rate of infectivity in a cell population and also produce high levels of gene expression in a short span of time (10-100-fold increase of expression within 24 h post-infection), so that the dynamics of expression between stably expressing and virally transduced cells would be expected to differ.

### 6.3 Discussion

Some animal galectins have been isolated and purified by biochemical procedures as molecules having hemagglutinin or other glyco-binding activity e.g. Galectins-1 and -3 (Blaser et al., 1998, Eur. J. Immunol. 28:2311-2319; Chadli et al., 1997, J. Neurochem., 68:1640-1647; Iglesias et al., 1998, Glycobiology 8: 59-65; Puch and Bhavanandan, 1999, Urology 53: 848-852). Others have been isolated in screens designed to identify molecules associated with specific biological phenomena such as carcinogenesis or differentiation, employing screens using expression cloning approaches e.g., Galectin-5, rat Galectin-8 and PCTA-1 (Gitt et al., 1998, J. Biol. Chem. 273: 2954-2960; Gitt et al., 1995, J. Biol. Chem. 270: 5032-5038; Hadari et al., 1995, J. Biol. Chem. 270: 3447-3453; Su et al., 1996, Proc. Natl. Acad. Sci. U.S.A. 93: 7252-7257). PCTA-1 was identified in two independent carcinoma related screens involving prostate cancer surface marker identification (Su et a., 1996, Proc. Natl. Acad. Sci. U.S.A. 93: 7252-7257) and in a lung cancer cDNA library expression screen (Bassen et al., 1999, Anticancer Res. 19: 5429-5433), respectively.

It may be noted that the isolation of rat galectin-8 occurred by chance during an expression screen with insulin-receptor substrate-1 antibody that should not have recognized this completely unrelated protein (Hadari et al., 1995, J. Biol. Chem. 270: 3447-3453). Our analysis with the Pro 1.5 antibody (Shen et al., 1994, J. Natl. Cancer Inst. 86: 91-98; Su et al., 1996, Proc. Natl. Acad. Sci. U.S.A. 93: 7252-7257) used to isolate PCTA-1 in the expression screen was however able to detect differences in protein level in cell lines and tissue sections between normal versus malignantly transformed human prostate samples (Su et al., 1996, Proc. Natl. Acad. Sci. U.S.A. 93: 7252-7257) implying specificity of recognition.

An additional connection to prostate cancer was provided by chromosomal localization of PCTA-1 to the 1q42.2-43 region of the human genome, shown by a recent European study to contain a putative predisposing gene for prostate cancer (Berthon et al., 1998, Proc. Natl. Acad. Sci. U.S.A. 93: 7252-7257). This region is distinct from the earlier predisposing locus thought to lie on 1q24-25.

Other genes including poly ADP-ribose polymerase and RAB4 (Ras-related GTP-binding protein) (Berthon et al., 1998, Proc. Natl. Acad. Sci. U.S.A. 93: 7252-7257) lie within this locus. It also harbors a fragile site (Feichtinger and Schmid, 1989, Hum. Genet. 83: 145-147), replication-error type-genetic instability locus (Nurty et al., 1994, Cancer Res. 54: 3983-3985) and was observed to translocate in glioblastomas (Li et al., 1995, Cancer Genet Cytogenet. 84: 46-50).

The CRDs of galectins comprise of an approximately 135 amino acid domain, which in PCTA-1 is separated by the so-called linker region. In the case of every galectin gene studied so far, the CRD is encoded by three exons (Gitt et al., 1998, J. Biol. Chem. 273: 2961-2970; Hadari et al., 1995, J. Biol. Chem. 270: 3447-3453), the middle one being most conserved and containing the codons encoding the sugar residue binding site. PCTA-1 is no exception in this respect, exons 3-5 and 8-10 contain the CRD encoding sequences. Comparable to the gene structure of Galectin-6, the linker region of PCTA-1 is encoded by two exons in the most prevalent mRNA species. The genomic organization of PCTA-1 varies from other Galectins including the other tandem repeat type (Galectins -4, -6 and -9) by alternate splicing of exons 7′ and 7″ causing variation in the length of the linker region (FIG. 1a). These are alternatively spliced into the ORF to extend the linker region by 126 bp (43 aa), 75 bp (25 aa) or 201 bp (68 aa) depending on whether 7′, 7″ or both combined together are spliced into the existing PCTA-1 ORF.

Deletion of the N-terminal domain of Galectin-3 impacts on its cellular localization (Mehul and Hughes, 1997, J. Cell. Sci. 110: 1169-1178). The linker region of tandem type Galectins are not homologous to the Galectin-3 N-terminal domain and the precise functionality of the additional isoforms await the production of reagents that can detect them specifically.

A significant extent of variation at the mRNA level also occurs at the 5′ and 3′ ends of the gene. Exon 1 is detectable in the mature transcript as an unspliced 828 bp or spliced 488 bp UTR region. Conformational analysis of this sequence for hairpin loops and other secondary conformations showed that a very high degree of stable secondary structure formation was possible (average free energy of 7100 Kcal) for both long and short forms of the 5′ UTR. The 5′ processing events were confirmed by performing primer extension analyses (FIG. 3B) that detected two bands of approximately 750 and 415 nt. The single transcription start site indicated in FIG. 3A is likely driven by a putative TATA-box containing promoter based on sequence homology and relative position of the relevant sequences.

Parallels can be drawn between the predicted value of free energy seen in the PCTA-1 5′ UTR and that observed for many growth regulatory genes (Willis, 1999, Int. J. Biochem. Cell. Biol. 31: 73-86). It is hypothesized that 5′ UTR secondary structure conformation acts as a further level of expression regulation by impeding passage of ribosomes and decreasing overall translatability of the mRNA (Willis, 1999, Int. J. Biochem. Cell. Biol. 31: 73-86). Specific cellular translation enhancing factors, that might play a role in overcoming the apparent translation block of such mRNA have been partially characterized and dysregulated activity of such translational enhancers has been hypothesized to play a role in transformation.

PCTA-1 protein levels might be modulated using a strategy commonly used by cells to fine tune expression of growth modulators (Willis, 1999, Int. J. Biochem. Cell. Biol. 31: 73-86). Alternative sites of polyadenylation and associated processing give rise to the three major isoforms of mRNA visible on Northern blots with PCTA-1 cDNA specific probes. The alternatively processed 3′ UTR is however transcribed from a single large exon 10. Commercial multiple tissue Northern blots made from polyadenylated RNA detected the three major species of transcripts of approximately 6.0, 2.6 and 1.6 kb (FIG. 4A-C, PCTA-1 ORF probe). The 2.6 and 1.6 kb transcripts are well represented in EST database sequences and has been independently confirmed by our own RT-PCR product sequence analysis. We reprobed one of the multiple tissue blots with a fragment specific for sequences downstream of the 3′ end of the 2.6 kb transcript. Since this detected a single major band of 6 kb, it confirmed the relationship between the three different transcripts as being alternately processed, truncated and polyadenylated variants of exon 10. When total RNA was used in Northern blot analyses two more transcripts of around 4 and 9 kb were visible (FIGS. 5A and 5F, i.e. Prostate and Melanoma ORF panels). Since these are not detectable using polyadenylated RNA they could represent processing intermediates of the primary transcript.

We attempted to determine if any specific pattern of expression existed for given isoforms in a normal or transformed cellular background. At the RNA level, PCTA-1 was expressed in both normal tissue (FIG. 4A-D, lanes 1±31; FIG. 5A-H, lane N) and transformed cell contexts (FIG. 5A-H, lanes D, L, P, C and H). Several independently prepared batches of RNA showed random variations in the relative amounts of different isoforms. The only reproducible observation was a higher level of expression of 1.6 and 2.6 kb transcripts in some cell lines from human prostate or melanoma (FIGS. 5A-D and 5F-G, respectively, lanes P and C). These lines have the capacity to spread metastatically in nude mouse tumor formation assays as opposed to the others. Previous reports, using RT-PCR assays (Bassen et al., 1999, Anticancer Res. 19: 5429-5433) have found a correlation with expression of isoforms containing the alternate exon encoded sequences and cellular transformation in tumor derived tissues. From results obtained by Northern blot analysis (FIGS. 5B and 5C, lane N, panels ALT1 and ALT2) it appears that normal prostate RNA apparently has a lower, but detectable level of message containing these sequences. The two additional transcripts of 4 and 9 kb are detected by a 3′ UTR probe downstream of the 3′ end of the 2.6 kb transcript. Since these are not visible on a blot made with polyA RNA we believe these could represent long lived partially processed forms of the primary transcript as described above. Again, cells with metastatic capacity in nude mice seem to contain higher levels of 9 kb transcript. FIG. 6 summarizes the several observed and predicted isoforms of PCTA-1 message based on information from gene structure, Northern blot, RT-PCR and EST sequence analyses.

Using live unfixed cells to determine cellular localization of GFP/PCTA-1 protein expression, we observed an essentially intracellular but extranuclear pattern of expression. This pattern was not uniformly disseminated throughout the cytosol, but showed micro-clustering reminiscent of organellar association (Bassen et al., 1999, Anticancer Res. 19: 5429-5433; Hadj Sahraoui et al., 1996, J. Cell. Biochem. 62: 529-542; Lutomski et al., 1997, Glycobiology 7: 1193-1199; Maldonado et al., 1999, Invest. Ophthalmol. Vis. Sci. 40: 2971-2977; Maquoi et al., 1997, Placenta 18: 433-439; Sarafian et al., 1998, Int. J. Cancer 75: 105-111; Su et al., 1996, Proc. Natl. Acad. Sci. U.S.A. 93: 7252-7257). The original study with the Pro 1.5 antibody implicated a cell surface localization of the antigen (Su et al., 1996, Proc. Natl. Acad. Sci. U.S.A. 93: 7252-7257). However, both Pro 1.5 and the Po-66 monoclonal antibody, which recognized an expression clone related to PCTA-1, in an independent screen, detected multiple bands in cellular protein extracts by Western blotting (Bassen et al., 1999, Anticancer Res. 19: 5429-5433; Su et a!, 1996, Proc. Natl. Acad. Sci. U.S.A. 93: 7252-7257). It is possible that the antibodies isolated in these immunological screens recognize additional polypeptides containing related epitopes.

Constitutive overexpression of PCTA-1 may be deleterious to cells in specific contexts such as contact independent growth. This finding could have implications in regulation of metastatic spread of cancer cells since significant growth inhibition is not observed in PCTA-1 overexpressing cells grown on normal tissue culture plasticware, only in soft agar assays. Two independent experimental strategies, one involving integration of the expression construct into genomic DNA (stable cell lines) and the other involving episomal expression (Adenovirus infected cells), showed inhibition of colony formation by full-length PCTA-1 overexpression. In addition, cell lines over-expressing a truncated version of PCTA-1, show notable levels of enhanced colony formation over control cell or those expressing the full-length ORF (30% and 70% respectively), implying that inhibition with wild-type protein is likely to be a genuine phenomenon. Overexpression of other galectins in cells or tissues have been similarly associated with opposing effects on growth, being stimulatory in some contexts (Bresalier et al., 1997, Cancer Res. 56: 4354-4357; Schoeppner et al., 1995, Cancer 75: 2818-2826) or inducing apoptosis in others (Perlllo et al., 1995, Nature 378: 736-739; Yang et al., 1996, Proc. Natl. Acad. Sci. U.S.A. 93: 6737-6742).

### 7. Example 1

### Full-Length PCTA-1 Suppresses Tumor Cell Growth, whereas Truncated PCTA-1 has Oncogenic Properties

Stable cell lines expressing full-length PCTA-1 (“B-PCTA-1”) or deletion mutant CR-1, encoding a truncated PCTA-1 protein (“T-PCTA-1”), were established using C8161 human metastatic melanoma cells. Compared to control, the B-PCTA-1-or T-PCTA-1-overexpressing lines showed no significant change in growth properties when plated on normal tissue culture plastic. However, when grown in the presence of the differentiation-promoting lymphokine, beta-interferon, a suppression in growth was observed for the line expressing B-PCTA-1, and, to a lesser extent, the T-PCTA-1-expressing line, compared to controls. The average cell count observed for the B-PCTA-1-expressing melanoma cells was 0.79×10⁵, compared to 1.05×10⁵ for cells expressing T-PCTA-1 and 3.99×10⁵ for control melanoma cells.

When these cells were tested for growth in soft agar of various densities, the following observations were made, with numbers indicating the average counts of total colonies (>50 cells) obtained/assay plate:

| Percent | B-PCTA-1, | T-PCTA-1, | |
|---|---|---|---|
| Agar | number of cells | number of cells | Control |
| 0.4 | 296 | 2282 | 1166 |
| 0.6 | 684 | 1533 | 1940 |
| 0.8 | 1022 | 1242 | 1738 |

There was an observed reversal in inhibition in colony formation of B-PCTA-1-expressing clones as the agar percentage increased, and a reduction in colony number for the T-PCTA-1-expressing clones. The ability to grow in higher concentrations of agar may reflect a more aggressive tumor phenotype.

The same cell lines were used in nude mouse tumorigenesis assays by injecting 1×10⁶ cells, combined with an equal volume of matrigel matrix, subcutaneously into the flanks of nude mice. Tumor formation at the site of injection was scored and volumes were measured as follows. The average tumor volume for control cells was 258 mm³. In contrast, the average tumor volume resulting from the injection of melanoma cells overexpressing B-PCTA-1 was 120 mm³, substantially smaller, and the average tumor volume resulting from the injection of melanoma cells overexpressing T-PCTA-1 was 575 mm³, substantially larger.

When the cell lines were injected into nude mice as above but in the absence of matrigel matrix, only CR-1 overexpressing lines formed palpable tumors that continued to grow progressively with time.

### 8. EXAMPLE

### Generation and Characterization of Various Transgenic Mouse Lines Expressing PCTA-1

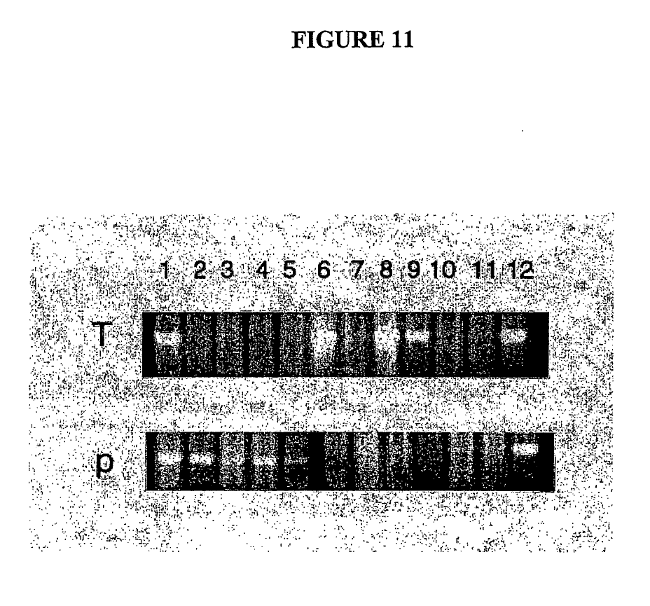

GENERATION AND CHARACTERIZATION OF VARIOUS TRANSGENIC MOUSE LINES EXPRESSING PCTA-1. A transgenic mouse line has been established to investigate the role of PCTA-1 in prostate cancer. In these studies, a BglII-xbaI fragment containing the full-length PCTA-1 cDNA (“B-PCTA-1”) was cloned into the BamHI/XbaI sites of the pcDEF3 expression vector. Expression of B-PCTA-1 from the resulting construct was confirmed through in vitro transfection studies. A MluI-AvrII fragment containing a full expression cassette comprising the human elongation factor 1α promoter, the B-PCTA-1 open reading frame, and the bovine growth hormone polyadenylation signal was excised from the pcDEF3 backbone, gel purified, and microinjected into mouse embryonic stem cells. The injected cells were then implanted into pseudopregnant female mice. Screening of offspring by Southern or PCR analysis confirms the presence of a randomly-integrated single copy of the PCTA-1 expression cassette (FIG. 11, lanes 1-5).

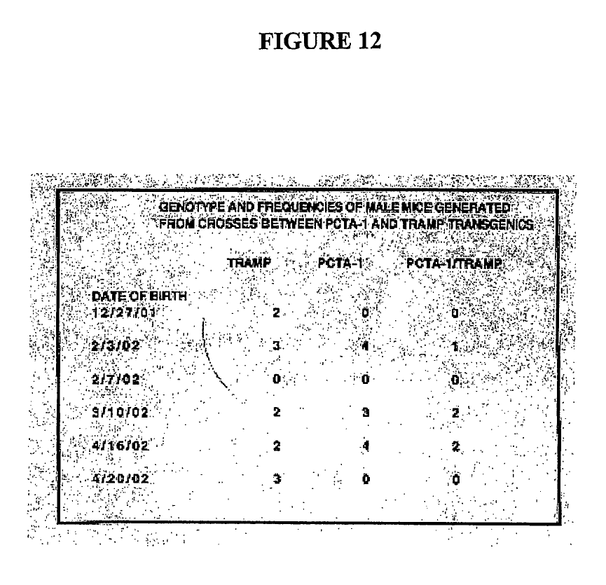

Based on the fact that the elongation factor 1α promoter is believed to be active in all cell and tissue types, PCTA-1 should be overexpressed in most organs and tissues of the transgenic mouse line. Thus far, the presence of the transgene is not associated with any overtly deleterious effects on normal growth, survival or fertility, as the frequency of progeny resulting from crosses of animals in which one or both parents were transgenic for the PCTA-1 gene resulted in the expected frequencies of wild-type and transgenic offspring (FIG. 12). The transgenic animals were of comparable size and survived to a similar age as wild-type littermates. These observations span several generations of crosses of this line (the oldest mice are 30 months of age). The fact that a deleterious phenotype has not been observed is consistent with the lack of an overt phenotypic effect of PCTA-1 expression on the growth and survival of cell lines derived from various types of human cancers (Gopalkrishnan et al., 2000, Oncogene, 19(38):4405-4416).

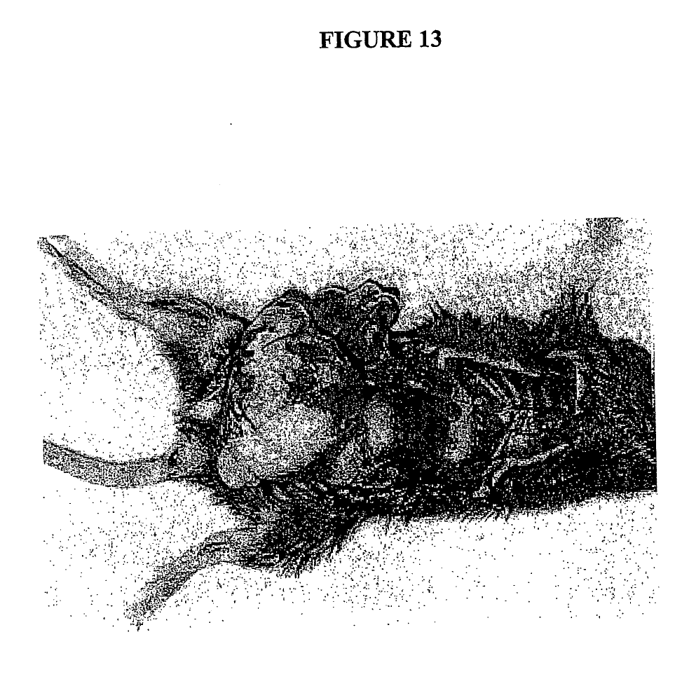

To assess the role of B-PCTA-1 expression on the development and spread of prostate tumors, the B-PCTA-1 transgenic mice were crossed onto the TRAMP (transgenic adenocarcinoma of the mouse prostate) transgenic mouse line. TRAMP transgenic mice represent an autochthonous mouse model of prostate cancer. TRAMP transgenic animals having mixed C56BL/6J backgrounds develop invasive adenocarcinomas that infiltrate the genitourinary tract and lower abdominal cavity over an average time span of 7 months (FIG. 13; see also Greenberg et al., 1995, Proc. Natl. Acad. Sci. USA 92(8 :3439-3443; Gingrich et al., 1996, Cancer Res. 56(18 :4096-4102; ). In general, tumorigenesis in mice is strain-dependent and FVB/N mice (Taketo et al., 1991, Proc. Natl. Acad. Sci. USA 88(6):2065-9) have been reported to show a faster development of TRAMP tumors compared to C56BL/6J.

Following a series of crosses in which B-PCTA-1 transgenic mice were crossed with TRAMP mice, litters containing doubly transgenic mice have been obtained (FIG. 11, lane 1), and the doubly transgenic animals were compared to age-matched littermates that are singly transgenic for TRAMP or PCTA-1. Palpable tumors have been detected in two singly transgenic TRAMP mice, while their doubly transgenic littermates are still free of detectable tumors, consistent with a tumor suppressive effect of B-PCTA-1.

Various publications and GenBank Accession Numbers have been referenced herein, the contents of which are hereby incorporated by reference in their entireties.

1-60. Cancelled

61. A non-human transgenic animal whose cells comprise the nucleic acid having the sequence of SEQ ID NO.:3.

62. The non-human transgenic animal of claim 61, wherein the nucleic acid having the sequence of SEQ ID NO.:3 is operably linked to a promoter.

63. The transgenic mouse of claim 62, wherein the promoter is the human elongation factor 1α promoter.

64. The non-human transgenic animal of any one of claim 61, wherein the cells of said non-human transgenic animal further comprise the nucleic acid encoding the SV40 T antigen.

65. The non-human transgenic animal of claim 64, wherein the nucleic acid encoding the SV40 T antigen is operably linked to a promoter.

66. The non-human transgenic animal of claim 65, wherein the promoter is the prostate cell-specific rat probasin promoter.

67. A non-human transgenic animal whose cells express a greater level of PCTA-1 protein as compared to the level of PCTA-1 protein expressed in a non-human non-transgenic animal of the same inbred strain.

68. A non-human transgenic animal having increased human PCTA-1 protein activity as compared to a non-human non-transgenic animal of the same inbred strain.

69. A non-human transgenic animal whose cells express a greater level of PCTA-1 mRNA as compared to the level of PCTA-1 mRNA expressed in a non-human non-transgenic animal of the same inbred strain.

70-73. Cancelled

74. The non-human transgenic animal of claim 62, wherein the cells of said non-human transgenic animal further comprise the nucleic acid encoding the SV40 T antigen.

75. The non-human transgenic animal of claim 63, wherein the cells of said non-human transgenic animal further comprise the nucleic acid encoding the SV40 T antigen.
